(19) United States
(12) Patent Application Publication
(10) Pub. No.: US 2021/0363590 A1
(43) Pub. Date: Nov. 25, 2021

(54) MOLECULAR GENE SIGNATURES AND METHODS OF USING SAME

(71) Applicant: NanoString Technologies, Inc., Seattle, WA (US)

(72) Inventors: Sarah WARREN, Seattle, WA (US); Patrick DANAHER, Seattle, WA (US)

(21) Appl. No.: 17/054,204

(22) PCT Filed: May 20, 2019
(86) PCT No.: PCT/US2019/033052

§ 371 Date: Nov. 10, 2020

Related U.S. Application Data
(60) Provisional application No. 62/747,853, filed on Oct. 19, 2018.
(60) Provisional application No. 62/674,285, filed on May 21, 2018.

(51) Int. Cl. C12Q 1/6886

(52) U.S. Cl. CPC C12Q 1/6886; C12Q 2600/158

(57) ABSTRACT

The invention provides methods of using expression levels of one or more cell gene signatures and/or combinations of cell gene signatures as selection criteria for selecting a patient having a cancer for treatment with a therapeutic. The invention further provides methods for selecting a patient having cancer who may benefit from a particular therapeutic, such as an immunotherapy and administering to the patient the immunotherapy to treat the cancer.

### CROSS-REFERENCE TO RELATED APPLICATIONS

This application claims priority to, and the benefit of, U.S. Provisional Application No. 62/674,285, filed May 21, 2018 and U.S. Provisional Application No. 62/747,853, filed Oct. 19, 2018. The contents of each of the aforementioned patent applications are incorporated herein by reference in their entireties.

### BACKGROUND OF THE INVENTION

The balance between effective anti-tumor immunity and immune evasion depends on diverse factors, including the abundance of various immune cell populations in the tumor microenvironment, the activities of those immune cells, tumor cell receptiveness to immune signaling, and microenvironment factors like nutrient availability and stroma. Many of these processes are onerous to measure, and no assay measures more than a small subset of them, slowing development of new immunotherapies and predictive biomarkers.

As gene expression in tumor specimens reflects activities within both tumor and immune cells, it promises a detailed readout of the tumor-immune interaction. However, gene expression results resist straightforward interpretation: even when we know the pathways a gene participates in, we often have little basis for linking its transcript's abundance to activity levels of a biological process. Thus a gene expression result, for example, “cytotoxicity genes are up-regulated in responders”, seldom establishes a more useful claim about biology, for example, “cytotoxic activity is higher in responders”.

Although, the project of linking gene expression to biological interpretation has been advanced by a growing literature using gene expression to measure the abundance of immune cell populations, cell type abundance provides an incomplete picture of the tumor microenvironment.

Hence, there is a current need to build a steady bridge from gene expression to biological interpretation in immune oncology, identifying genes whose expression appears to track a specific biological process and incorporating these genes into signatures measuring the key biology of immune oncology. In addition, more than the presence of immune cells, there is a need to measure the activities of those cells, as well the diverse interactions between tumor cells and the immune system. For example, immune processes like cytotoxicity, antigen presentation and interferon gamma signaling may be more important to measure than the cell types capable of performing them, and cell type measurements are blind to the non-immune-intrinsic processes that shape the tumor-immune interaction, such as nutrient availability, angiogenesis, and antigen presentation and JAK-STAT signaling within tumor cells.

The present invention addresses the above-mentioned needs and expands the window gene expression provides into the tumor-immune interaction, by providing signatures of the various tumor- and immune-intrinsic processes driving immune response and escape.

### SUMMARY OF THE INVENTION

In one aspect, the present disclosure relates to a method of selecting treatment for a cancer patient in need thereof, comprising determining the expression level of any combination of any gene, or groups of genes, or combination of genes or of groups of genes, recited in any gene signature herein in any form.

In one aspect, the invention relates to a method of selecting a treatment for a cancer patient in need thereof comprising determining the expression level of one or more genes in at least one of the signatures (a)-(q) in a biological sample obtained from the patient:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
   wherein a change in the level of expression of one or more of the genes in the at least one gene signature identifies a patient for treatment. In another aspect, the method comprises of selecting a treatment for a cancer patient in need thereof comprising determining the expression level of one or more genes, or groups of genes, or combination of genes or of groups of genes, recited in signatures (a)-(q) in a biological sample obtained from the patient, wherein a change in the level of expression of one or more genes, or groups of genes, or combination of genes or of groups of genes, in the gene signatures (a)-(q) identifies a patient for treatment.

In a related aspect, the invention relates to a method of selecting a subject having cancer for treatment with a therapeutic comprising determining the expression level of one or more genes in at least one of the signatures (a)-(q) in a biological sample obtained from the subject:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
   wherein a change in the level of expression of one or more of the genes in the at least one of the gene signatures (a)-(q) identifies a subject for treatment with a therapeutic. In another aspect, the method comprises of selecting a subject having cancer for treatment with a therapeutic comprising determining the expression level of one or more genes, or groups of genes, or combination of genes or of groups of genes, recited in signatures (a)-(q) in a biological sample obtained from the patient, wherein a change in the level of expression of one or more of the genes, or groups of genes, or combination of genes or of groups of genes, in the gene signatures (a)-(q) identifies a subject for treatment with a therapeutic.

In a related aspect, the invention relates to a method of identifying a subject having cancer as likely to respond to treatment with a therapeutic comprising determining the expression level of one or more genes in at least one of the signatures (a)-(q) in a biological sample obtained from the subject:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
   wherein a change in the level of expression of one or more of the genes in the at least one of the gene signatures (a)-(q) identifies a patient likely to respond to treatment with a therapeutic. In another aspect, the method comprises identifying a subject having cancer as likely to respond to treatment with a therapeutic comprising determining the expression level of one or more genes, or groups of genes, or combination of genes or of groups of genes, recited in signatures (a)-(q) in a biological sample obtained from the patient, wherein a change in the level of expression of one or more genes, or groups of genes, or combination of genes or of groups of genes, in the gene signatures (a)-(q) identifies a patient likely to respond to treatment with a therapeutic.

In a related aspect, the invention relates to a method for monitoring pharmacodynamic activity of a cancer treatment in a subject, comprising:

(i) measuring the expression level of one or more of the genes in at least one of the signatures (a)-(q) in a biological sample obtained from the subject, wherein the subject has been treated with a therapeutic
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6; and
   (ii) determining the treatment as demonstrating pharmacodynamic activity based on the expression level of the one or more genes in the sample obtained from the subject, wherein an increased or decreased expression level of the one or more genes in the sample obtained from the subject indicates pharmacodynamic activity of the therapeutic. In another aspect, the invention relates to a method for monitoring pharmacodynamic activity of a cancer treatment in a subject, comprising:
   (i) measuring the expression level of one or more genes, or groups of genes, or combination of genes or of groups of genes, in the signatures (a)-(q) in a biological sample obtained from the subject, wherein the subject has been treated with a therapeutic, and
   (ii) determining the treatment as demonstrating pharmacodynamic activity based on the expression level of the of one or more genes, or groups of genes, or combination of genes or of groups of genes, in the sample obtained from the subject, wherein an increased or decreased expression level of the one or more genes, or groups of genes, or combination of genes or of groups of genes, in the sample obtained from the subject indicates pharmacodynamic activity of the therapeutic.

In another related aspect, the invention features a method of selecting a patient having cancer for treatment with a therapeutic, the method comprising determining the expression level of a cell gene signature in a biological sample obtained from the patient, the cell gene signature comprising one or more of the following genes (e.g., 1, 2, 3, 4, 5, 6, 7, 8, 9, 10, 11, 12, 13, 14, 15, 16, 17, or more of the genes selected from the gene signatures in Table 1).

In one embodiment, a method provided herein is carried out using any combination of genes or any combination of gene signatures set forth in Table 1. In another embodiment, a method provided herein is carried out using any combination or permutation (in any order) of any one or more of the 17 gene signatures set forth in Table 1. In some embodiments, the invention features a method of selecting a patient having cancer for treatment with a therapeutic, the method comprising determining the expression level of a cell gene signature in a biological sample obtained from the patient, the cell gene signature comprising one or more of the genes in at least one of the signatures recited in Table 1 herein, wherein a change in the level of expression of the one or more genes in the cell gene signature relative to a median level identifies a patient for treatment with a therapeutic.

In some embodiments, the invention features a method of selecting a patient having cancer for treatment with an immunotherapy, the method comprising determining the expression level of an cell gene signature in a biological sample obtained from the patient, the cell gene signature comprising one or more of the genes in at least one of the signatures recited in Table 1 herein, wherein a change in the level of expression of the one or more genes in the cell gene signature relative to a median level identifies a patient for treatment with an immunotherapy.

In one embodiment, the method of the present invention further comprises the step of informing the patient that they have an increased likelihood of being responsive to the therapeutic. In another embodiment, the method further comprises the step of providing a recommendation to the patient for a particular therapeutic. In some embodiments, the method further comprises the step of administering a targeted therapy to the patient if it is determined that the patient may benefit from the therapeutic.

In some embodiments, the method further comprises the step of informing the patient that they have an increased likelihood of being responsive to an immunotherapy. In other embodiments, the method further comprises the step of providing a recommendation to the patient for a particular immunotherapy. In some embodiments, the method further comprises the step of administering an immunotherapy to the patient if it is determined that the patient may benefit from the immunotherapy. In other embodiments, the immunotherapy is an activating immunotherapy or a suppressing immunotherapy.

In one embodiment, an increase in expression level of one or more of the genes recited in Table 1 indicates that the patient is likely to benefit from an activating immunotherapy. In some embodiments, the activating immunotherapy comprises an agonist of at least one or more genes from one or more gene signature recited in Table 1. In some embodiments, where the patient is likely to benefit from a suppressing immunotherapy, the suppressing immunotherapy comprises an antagonist of at least one or more genes from at least one or more gene signature recited in Table 1. In one embodiment, the activating immunotherapy or suppressing immunotherapy comprises an agonist or antagonist of at least at one or more genes selected from the proliferation, lymphoid, cytotoxicity, myeloid, myeloid inflammation, interferon-gamma, interferon-downstream, MHC2 or a combination thereof gene signatures from Table 1.

In one embodiment, the expression level of one or more genes recited in Table 1 is linked to a biological process described herein, such as a cancer, or a condition or disease. In some embodiments, the expression level of one or more genes listed in at least the lymphoid cell gene signature recited in Table 1 is correlated with the presence of lymphoid cells in the tumor or in the tumor microenvironment. In some embodiments, the expression level of one or more genes listed in at least the myeloid cell gene signature recited in Table 1 is correlated with the presence of myeloid cells in the tumor or in the tumor microenvironment. In some embodiments, the expression level of one or more genes listed in at least the cell proliferation gene signature recited in Table 1 is correlated with cellular proliferation. In some embodiments, the expression level of one or more genes listed in at least the lymphoid cell gene signature recited in Table 1 is correlated with the presence of B cells in the tumor microenvironment. In some embodiments, the expression level of one or more genes listed in at least the lymphoid cell gene signature recited in Table 1 is correlated with the presence of Natural Killer cells in the tumor microenvironment. In some embodiments, the expression level of one or more of genes listed in at least the lymphoid cell gene signature recited in Table 1 is correlated with the presence of costimulatory ligands in the tumor microenvironment. In some embodiments, the expression level of one or more of genes listed in at least the lymphoid cell gene signature recited in Table 1 is correlated with the presence of costimulatory receptors in the tumor microenvironment. In some embodiments, the expression level of one or more of genes listed in at least the lymphoid cell gene signature recited in Table 1 is correlated with the presence of T cells in the tumor microenvironment. In some embodiments, the expression level of one or more genes listed in at least the myeloid cell gene signature listed in Table 1 is correlated with the presence of macrophage cells in the tumor microenvironment.

In some embodiments, the expression level of one or more genes listed in at least the myeloid cell gene signature recited in Table 1 is correlated with the presence of M2 macrophage cells in the tumor microenvironment. In some embodiments, the expression level of one or more of genes listed in at least the myeloid cell gene signature, the myeloid inflammation gene signature or the inflammatory chemokines gene signature recited in Table 1 is correlated with the presence of inflammatory cells in the tumor microenvironment. In some embodiments, the expression level of one or more of genes listed in at least the myeloid cell gene signature or the lymphoid cell gene signature recited in Table 1 is correlated with the presence of T cell immune blockers in the tumor microenvironment. In some embodiments, the expression level of one or more of genes listed in at least the myeloid cell gene signature or the lymphoid cell gene signature recited in Table 1 is correlated to the presence of antigen presenting cell (APC) immune blockers in the tumor microenvironment. In some embodiments, the expression level of one or more of genes listed in at least the interferon gamma gene signature or the lymphoid cell gene signature recited in Table 1 is correlated with T cell chemotaxis. In some embodiments, the expression level of one or more of genes listed in at least the antigen processing machinery (APM) cell or the immunoproteosome gene signature recited in Table 1 is correlated with the presence of antigen processing in the tumor microenvironment. In some embodiments, the expression level of one or more of genes listed in at least the cytotoxicity cell gene signature recited in Table 1 is correlated with cytolytic activity and/or the presence of cytolytic cells in the tumor microenvironment. In some embodiments, the expression level of one or more of genes listed in at least the stroma cell gene signature recited in Table 1 is correlated with the presence of active fibroblasts in the tumor microenvironment. In some embodiments, the expression level of one or more of genes listed in at least the MAGE gene signature recited in Table 1 is correlated with the presence of MAGE-class antigens on the tumor surface. In some embodiments, the expression level of one or more of genes listed in at least the interferon gamma gene signature is correlated with T cell chemotaxis.

In some embodiments, the expression level of one or more of genes listed in at least the apoptosis gene signature recited in Table 1 is correlated with the presence of cells undergoing apoptosis in the tumor or tumor microenvironment In some embodiments, the expression level of one or more of genes listed in at least the hypoxia gene signature recited in Table 1 is correlated with the abundance of cells initiating angiogenesis and regulating cellular metabolism to overcome hypoxia. In some embodiments, the expression level of one or more of genes listed in the glycolytic activity gene signature recited in Table 1 is correlated with the amount of glycolysis in a tumor. In some embodiments, the expression level of one or more of genes listed in at least the interferon-downstream gene signature recited in Table 1 is correlated with the amount of the tumor's signaling pathway activity induced by exposure to interferons.

In other embodiments of any of the above methods, the expression level is one or more of a gene listed in a gene signature recited in Table 1 is determined.

In some embodiments of any of the above methods, the method further comprises determining the ratio of expression level of one or more genes listed in at least one gene signature recited in Table 1 relative to a medial level.

In some embodiments of any of the above methods, the method is carried out prior to administering the targeted therapy in order to provide a patient with a pre-administration prognosis for response. In some embodiments of any of the above methods, the method is carried out prior to administering the therapeutic in order to provide a patient with a pre-administration prognosis for response.

In some embodiments of any of the above methods, the cancer is a cancer is adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma, breast cancer, lung cancer, lymphoma, melanoma, liver cancer, colorectal cancer, ovarian cancer, bladder cancer, renal cancer or gastric cancer, neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or a cervical cancer.

In some embodiments of any of the above methods, expression of the cell gene signature in the biological sample obtained from the patient is detected by measuring mRNA.

In some embodiments of any of the above methods, expression of the cell gene signature in the biological sample obtained from the patient is detected by measuring protein levels.

The methods of the present disclosure can further comprise administering to the subject at least one therapeutically effective amount of at least one treatment. The at least one treatment can comprise anti-cancer therapy. The at least one treatment can comprise immunotherapy. Immunotherapy can comprise activating immunotherapy, suppressing immunotherapy, or a combination of an activating and a suppressing immunotherapy. Immunotherapy can comprise the administration of at least one therapeutically effective amount of at least one checkpoint inhibitor, at least one therapeutically effective amount of at least one chimeric antigen receptor T-cell therapy, at least one therapeutically effective amount of at least one oncolytic vaccine, at least one therapeutically effective amount of at least one cytokine agonist, at least one therapeutically effective amount of at least one cytokine antagonist, or any combination thereof.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Other features and advantages of the present invention will become apparent from the following detailed description examples and figures. It should be understood, however, that the detailed description and the specific examples while indicating embodiments of the invention are given by way of illustration only, since various changes and modifications within the spirit and scope of the invention will become apparent to those skilled in the art from this detailed description.

### BRIEF DESCRIPTION OF THE DRAWINGS

Any of the above aspects and embodiments can be combined with any other aspect or embodiment as disclosed here in the Summary and/or Detailed Description sections.

FIG. 1 illustrates the strength of co-expression in each signature's gene set.

FIG. 2 illustrates the effectiveness of predictor training using single genes vs. our signatures in an immunotherapy dataset with 8 responders and 34 non-responders.

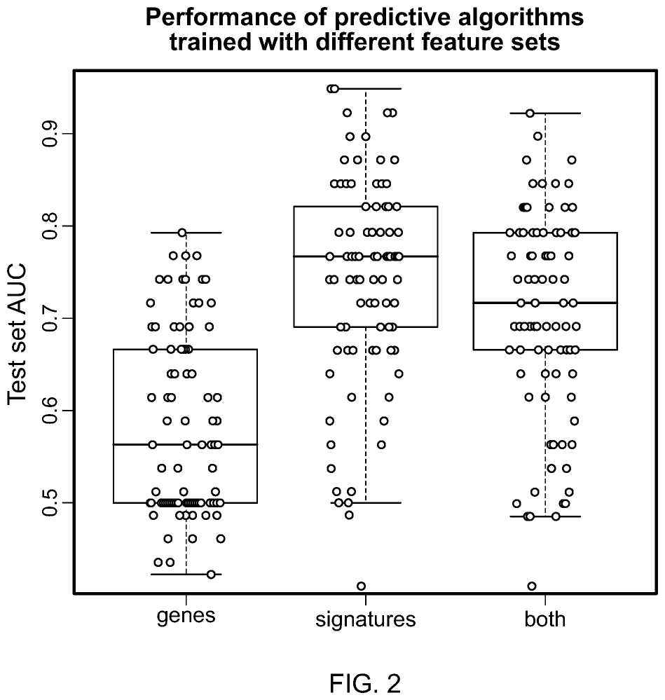

FIG. 3 illustrates the association between immune signatures and response to anti-PD1 immunotherapy. Boxes show average log₂ fold-changes between responders and non-responders; bars show 95% confidence intervals.

FIG. 4 illustrates results of models predicting response from pairs of signatures. Color denotes −log₁₀ p-values. Signature pairs with p-values above 0.05 are white.

### DETAILED DESCRIPTION OF THE INVENTION

In many cases, a gene signature that merely averages a collection of biologically plausible genes will successfully measure the intended biological process. However, many biological processes are governed not by modulating mRNA abundance but rather protein abundance, binding or location and hence, attempts to measure these processes with gene expression will produce misleading results. Therefore, biological knowledge alone is an unsuitable basis for gene signatures. The present invention provides a bridge from gene expression to biological interpretation in immune oncology, identifying genes whose expression track a specific biological process and incorporating these genes into signatures measuring the key biology of immune oncology.

Accordingly, the invention provides methods for selecting a patient having cancer (e.g., bladder cancer, breast cancer, colorectal cancer, gastric cancer, liver cancer, melanoma, lung cancer (e.g., non-small cell lung carcinoma), ovarian cancer, or renal cell carcinoma) for treatment with an immunotherapy by determining the expression level of one or more cell gene signatures, and comparing this level of expression to the median level of expression of the one or more cell gene signatures. Detection of increased expression of the one or more cell gene signatures relative to a median level (i.e., higher expression of the one or more cell gene signatures relative to the median level in the cancer type) identifies the patient for treatment with an immunotherapy. The invention also provides methods for treating a patient having cancer (e.g., bladder cancer, breast cancer, colorectal cancer, gastric cancer, liver cancer, melanoma, lung cancer (e.g., non-small cell lung carcinoma), ovarian cancer, or renal cell carcinoma) who may benefit from a therapeutic described herein. An example of a therapeutic described herein can be administering an activating immunotherapy or a suppressing immunotherapy alone or in combination with a chemotherapy regimen and/or other anti-cancer therapy regimen by determining the expression level of one or more cell gene signatures in the patient.

### Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

The term “antagonist” is used in the broadest sense, and includes any molecule that partially or fully blocks, inhibits, interferes, or neutralizes a normal biological activity of a native polypeptide disclosed herein (e.g., an immune cell receptor or ligand, such as CTLA-4, PD-1, TIM-3, BTLA, VISTA, LAG-3, B7H4, CD96, TIGIT, or CD226), either by decreasing transcription or translation of the nucleic acid encoding the native polypeptide, or by inhibiting or blocking the native polypeptide activity, or both. It will be understood by one of ordinary skill in the art that, in some instances, an antagonist may antagonize one activity of the native polypeptide without affecting another activity of the native polypeptide. It will also be understood by one of ordinary skill in the art that, in some instances, an antagonist may be a therapeutic agent that is considered an activating or suppressing immunotherapy depending on the native polypeptide that it binds, interacts, or associates with. Examples of antagonists include, but are not limited to, antisense polynucleotides, interfering RNAs, catalytic RNAs, RNA-DNA chimeras, native polypeptide-specific aptamers, antibodies, antigen-binding fragments of antibodies, native polypeptide-binding small molecules, native polypeptide-binding peptides, and other peptides that specifically bind the native polypeptide (including, but not limited to native polypeptide-binding fragments of one or more native polypeptide ligands, optionally fused to one or more additional domains), such that the interaction between the antagonist and the native polypeptide results in a reduction or cessation of native polypeptide activity or expression.

In a similar manner, the term “agonist” is used in the broadest sense and includes any molecule that mimics, promotes, stimulates, or enhances a normal biological activity of a native polypeptide disclosed herein (e.g., an immune cell receptor or ligand, such as GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof), by increasing transcription or translation of the nucleic acid encoding the native polypeptide, and/or by inhibiting or blocking activity of a molecule that inhibits the expression or activity of the native polypeptide, and/or by enhancing normal native polypeptide activity (including, but not limited to, enhancing the stability of the native polypeptide, or enhancing binding of the native polypeptide to one or more target ligands). It will be understood by one of ordinary skill in the art that, in some instances, an agonist may agonize one activity of the native polypeptide without affecting another activity of the native polypeptide. It will also be understood by one of ordinary skill in the art that, in some instances, an agonist may be a therapeutic agent that is considered an activating or suppressing immunotherapy depending on the native polypeptide that it binds, interacts, or associates with. The agonist can be selected from an antibody, an antigen-binding fragment, an aptamer, an interfering RNA, a small molecule, a peptide, an antisense molecule, and another binding polypeptide. In another example, the agonist can be a polynucleotide selected from an aptamer, interfering RNA, or antisense molecule that interferes with the transcription and/or translation of a native polypeptide-inhibitory molecule.

Methods for identifying agonists or antagonists of a polypeptide may comprise contacting a polypeptide with a candidate agonist or antagonist molecule and measuring a detectable change in one or more biological activities normally associated with the polypeptide.

The term “activating immunotherapy” refers to the use of a therapeutic agent that induces, enhances, or promotes an immune response, including, e.g., a T cell response. The term “suppressing immunotherapy” refers to the use of a therapeutic agent that interferes with, suppresses, or inhibits an immune response, including, e.g., a T cell response.

“Human effector cells” refer to leukocytes that express one or more FcRs and perform effector functions. In certain embodiments, the cells express at least FcyRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Regulatory T cells (T_{reg})” refer to a subset of helper T cells that play a role in inhibition of self-reactive immune responses and are often found in sites of chronic inflammation such as in tumor tissue, in certain embodiments, T_{regs} are defined phenotypically by high cell surface expression of CD25, CLTA4, GITR, and neuropilin-1 and are under the control of transcription factor FOXP3. In other embodiments, T_{regs} perform their suppressive function on activated T cells through contact-dependent mechanisms and cytokine production. In some embodiments, T_{regs} also modulate immune responses by direct interaction with ligands on dendritic cells (DC), such as, e.g., CTLA4 interaction with B7 molecules on DC that elicits the induction of indoleamine 2, 3-dioxygenase (IDO).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (MA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

The term “biological sample” or “sample” as used herein includes, but is not limited to, blood, serum, plasma, sputum, tissue biopsies, tumor tissue, and nasal samples including nasal swabs or nasal polyps. In one embodiment, the biological sample is obtained from the subject before a therapy or therapeutic described herein is administered to the subject. In another embodiment, the biological sample is obtained from the subject after the therapy or therapeutic described herein is administered to the subject. In one particular embodiment, the biological sample is tumor tissue. In another particular embodiment, the biological sample is blood. In other embodiment, the sample is plasma, cerebrospinal fluid (CSF), saliva, or any bodily fluid.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

An “advanced” cancer is one which has spread outside the site or organ of origin, either by local invasion or metastasis.

A “refractory” cancer is one which progresses even though an anti-tumor agent, such as a chemotherapeutic agent, is being administered to the cancer patient. An example of a refractory cancer is one which is platinum refractory.

A “recurrent” cancer is one which has regrown, either at the initial site or at a distant site, after a response to initial therapy.

By “platinum-resistant” cancer is meant cancer in a patient that has progressed while the patient was receiving platinum-based chemotherapy or cancer in a patient that has progressed within, e.g., 12 months (for instance, within 6 months) after the completion of platinum-based chemotherapy. Such a cancer can be said to have or exhibit “platinum-resistance.”

By “chemotherapy-resistant” cancer is meant cancer in a patient that has progressed while the patient is receiving a chemotherapy regimen or cancer in a patient that has progressed within, e.g., 12 months (for instance, within 6 months) after the completion of a chemotherapy regimen. Such a cancer can be said to have or exhibit “chemotherapy-resistance.”

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant. The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgGI, IgG2, IgG3, IgG4, IgA1, and IgA2. The heavy chain constant domains that correspond to the different classes of immunoglobulins are called α, δ, ε, γ, and μ, respectively.

A “chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5a-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustdnitrosoureas such as carmustine, chlorozotocin, fotemustine, lomustine, nimustine, and ranimnustine; antibiotics such as the enediyne antibiotics (e.g., calichmicin, especially calicheamicin γ1 1 and calicheamicin ω1 1 (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, III.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-1 1; topoisomerase inhibitor RFS 2000; difluoromethylornithine (DMFO); retinoids such as retinoic acid; and pharmaceutically acceptable salts, acids and derivatives of any of the above.

A chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY1 17018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rlL-2; a topoisomerase 1 inhibitor such as LURTOTECAN®; ABARELIX® rmRH; and (ix) pharmaceutically acceptable salts, acids and derivatives of any of the above.

A chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idee), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

A chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-1 1 F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.1 1, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582; 5,457,105; 5,475,001; 5,654,307; 5,679,683; 6,084,095; 6,265,410; 6,455,534; 6,521,620; 6,596,726; 6,713,484; 5,770,599; 6,140,332; 5,866,572; 6,399,602; 6,344,459; 6,602,863; 6,391,874; 6,344,455; 5,760,041; 6,002,008; and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1 H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as Cl-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1 C1 1 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1 999/06378 (Warner Lambert); WO 1 999/06396 (Warner Lambert); WO 1 996/30347 (Pfizer, Inc); WO 1 996/33978 (Zeneca); WO 1 996/3397 (Zeneca) and WO 1 996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

By “platinum-based chemotherapeutic agent” or “platin” is meant an antineoplastic drug that is a coordination complex of platinum. Examples of platinum-based chemotherapeutic agents include carboplatin, cisplatin, satraplatin, picoplatin, nedaplatin, triplatin, lipoplatin, and oxaliplatinum.

By “platinum-based chemotherapy” is meant therapy with one or more platinum-based chemotherapeutic agent, optionally in combination with one or more other chemotherapeutic agents.

By “correlate” or “correlation” or grammatical equivalents is meant comparing, in any way, the performance and/or results of a first analysis or protocol with the performance and/or results of a second analysis or protocol. For example, one may use the results of a first analysis or protocol to determine the outcome or result of a second analysis or protocol. Or one may use the results of a first analysis or protocol to determine whether a second analysis or protocol should be performed. For example, with respect to the embodiment of gene expression analysis or protocol, one may use the results of the gene expression analysis or protocol to determine whether a specific immune cell type or subset is present.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Enhancing T cell function” means to induce, cause or stimulate an effector or memory T cell to have a renewed, sustained or amplified biological function. Examples of enhancing T cell function include: increased secretion of γ-interferon from CD8 effector T cells, increased secretion of γ-interferon from CD4+ memory and/or effector T cells, increased proliferation of CD4+ effector and/or memory T cells, increased proliferation of CD8 effector T cells, increased antigen responsiveness (e.g., clearance), relative to such levels before the intervention. In one embodiment, the level of enhancement is at least 50%, alternatively 60%, 70%, 80%, 90%, 100%, 120%, 150%, 200%. The manner of measuring this enhancement is known to one of ordinary skill in the art.

A sample, cell, tumor, or cancer which “expresses” one or more cell gene signatures at an increased expression level relative to a median level of expression (e.g., the median level of expression of the one or more cell gene signatures in the type of cancer (or in a cancer type, wherein the “cancer type” is meant to include cancerous cells (e.g., tumor cells, tumor tissues) as well as non-cancerous cells (e.g., stromal cells, stromal tissues) that surround the cancerous/tumor environment) is one in which the expression level of one or more cell gene signatures is considered to be a “high cell gene signature expression level” to a skilled person for that type of cancer. Generally, such a level will be in the range from about 50% up to about 100% or more (e.g., 50%, 55%, 60%, 65%, 70%, 75%, 80%, 85%, 90%, 95%, 100%, or more) relative to cell gene signature levels in a population of samples, cells, tumors, or cancers of the same cancer type. For instance, the population that is used to arrive at the median expression level may be particular cancer samples (e.g., adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer) generally, or subgroupings thereof, such as chemotherapy-resistant cancer, platinum-resistant cancer, as well as advanced, refractory, or recurrent cancer samples.

By “determining the expression level” used in reference to a particular biomarker (e.g., one or more genes from the cell gene signatures), means expression of the biomarker(s) (e.g., one or more genes from the cell gene signatures) in a cancer-associated biological environment (e.g., expression of the biomarker(s) in the tumor cells), tumor-associated cells (e.g., tumor-associated stromal cells), as determined using a diagnostic test, any of the detection methods described herein, or the similar. In one embodiment, expression of the one or more genes in the biological sample form the patient is determined by measuring mRNA. In other embodiments, expression of the one or more genes in the biological sample form the patient is determined by measuring mRNA in plasma, by measuring mRNA in tissue, by measuring mRNA in FFPE tissue, by measuring protein levels, by measuring protein levels in plasma, by measuring protein levels in tissue, by measuring protein levels in FFPE tissue or a combination thereof.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2(L2)-FR3-H3(L3)-FR4. In some embodiments, an antibody used herein comprises a human consensus framework.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al, Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al, supra. In one embodiment, for the VH, the subgroup is subgroup III as in Kabat et al, supra. A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (HI, H2, H3), and three in the VL (LI, L2, L3). HVRs generally comprise amino acid residues from the hypervariable loops and/or from the “complementarity determining regions” (CDRs), the latter typically being of highest sequence variability and/or involved in antigen recognition. An HVR region as used herein comprise any number of residues located within positions 24-36 (for HVRL1), 46-56 (for HVRL2), 89-97 (for HVRL3), 26-35B (for HVRH1), 47-65 (for HVRH2), and 93-102 (for HVRH3).

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance. “Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with a CD28, OX40, GITR, CD137, CD27, ICOS, HVEM, NKG2D, MICA, or 2B4 agonist or treatment with a CTLA-4, PD-1 axis, TIM-3, BTLA, VISTA, LAG-3, B7H4, CD96, TIGIT, or CD226 antagonist.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). F or review of methods for assessment of antibody purity, see, e.g., Flatman et al, J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. “Isolated nucleic acid encoding an anti-target antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used according to the methods provided herein may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Patient response” or “response” (and grammatical variations thereof) can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

By “radiation therapy” or “radiation” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

The term “small molecule” refers to an organic molecule having a molecular weight between 50 Daltons to 2500 Daltons.

The terms “cell gene signature” refers to any one or a combination or sub-combination of the genes set forth in Table 1. Such sub-combinations of these genes are sometimes referred to as “gene sets,” and exemplary “gene sets” are set forth in Tables 2-17. The term “immune cell signature” refers to the gene expression pattern of a cell gene signature in a patient that correlates with the presence of an immune cell subtype (e.g., T effector cells, T regulatory cells, B cells, NK cells, myeloid cells, Th17 cells, inflammatory cells, T cell immune blockers, and antigen presenting cell (APC) immune blockers). Each individual gene or member of a cell gene signature is a “cell signature gene.” Further, each individual gene or member of an immune cell gene signature is an “immune cell signature gene.” These genes include, without limitation the genes from the lymphoid gene signature set in Table 1: CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48, ICOS or for example, the genes from the myeloid gene signature set in Table 1: ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1, CEBPB.

The term “PD1-axis antagonist” refers to a molecule that inhibits the interaction of a PD-1 axis binding partner with either one or more of its binding partner, so as to remove T cell dysfunction resulting from signaling on the PD-1 signaling axis-with a result being to restore or enhance T cell function (e.g., proliferation, cytokine production, target cell killing). As used herein, a PD-1 axis antagonist includes a PD-1 binding antagonist, a PD-L1 binding antagonist, and a PD-L2 binding antagonist.

“Survival” refers to the patient remaining alive, and includes overall survival as well as progression free survival.

“Overall survival” refers to the patient remaining alive for a defined period of time, such as 1 year, 5 years, etc. from the time of diagnosis or treatment.

The phrase “progression-free survival” in the context of the present invention refers to the length of time during and after treatment during which, according to the assessment of the treating physician or investigator, a patient's disease does not become worse, i.e., does not progress. As the skilled person will appreciate, a patient's progression-free survival is improved or enhanced if the patient experiences a longer length of time during which the disease does not progress as compared to the average or mean progression free survival time of a control group of similarly situated patients.

By “standard of care” herein is intended the anti-tumor/anti-cancer, anti-condition or anti-disease agent or agents that are routinely used to treat a particular form of cancer, condition or disease.

The terms “therapeutically effective amount” or “effective amount” refer to an amount of a drug effective to treat a cancer, condition or disease in the patient. For example, with respect to cancer, the effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), improve survival (including overall survival and progression free survival) and/or improve one or more symptoms of cancer (e.g. as assessed by FOSI). Most preferably, the therapeutically effective amount of the drug is effective to improve progression free survival (PFS) and/or overall survival (OS).

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, methods and compositions of the invention are useful in attempts to delay development of a disease or disorder.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al, J. Immunol. 1 50:880-887 (1993); Clarkson et al, Nature 352:624-628 (1991).

Methods of Prognosis and Detection

The present invention relates to the identification, selection, and use of biomarkers of cancer (e.g., adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma, breast cancer, lung cancer, lymphoma, melanoma, liver cancer, colorectal cancer, ovarian cancer, bladder cancer, renal cancer or gastric cancer, neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer) that are correlated with an immune cell subtype (e.g., T effector cells, T regulatory cells, B cells, NK cells, myeloid cells, inflammatory cells, T cell immune blockers, antigen presenting cell (APC) immune blockers). In this respect, the invention relates to analysis of expression profile(s) in samples from patients with cancer involved in tumor immunity and the use of these biomarkers in selecting patients for treatment with immunotherapy. The biomarkers of the invention are listed herein, e.g., in Table 1. Gene signature sets

TABLE 1
Gene Signature Sets

| Gene Signature | Gene Signature Gene Members |
|---|---|
| Proliferation | MKI67, CEP55, KIF2C, MELK, CENPF, EXO1, |
| | ANLN, RRM2, UBE2C, CCNB1, CDC20 |
| Stroma | FAP, COL6A3, ADAM12, OLFML2B, PDGFRB, |
| | LRRC32 |
| Lymphoid | CXCL10, CXCR3, CX3CL1, PRF1, GZMK, |
| | GZMB, CD27, IL2RG, KLRK1, CTLA4, GZMH, |
| | CD3D, KLRB1, KLRD1, LCK, CD5, IRF4, CD8A, |
| | CD38, EOMES, GZMM, GNLY, IFITM1, IDO1, |
| | MS4A1, GZMA, CD2, CD3E, CD3G, CD40LG, |
| | CD6, CD7, CD79A, CD8B, CXCL11, CXCL13, |
| | CXCL9, HLA-DOB, IFNG, LAG3, LY9, PDCD1, |
| | TBX21, TIGIT, ZAP70, SLAMF7, CD96, PVR, |
| | STAT1, JAK1, JAK2, STAT2, IRF9, IGF2R, CD48, |
| | ICOS |
| Myeloid | ITGAM, TLR4, IL1B, CSF1R, CSF3R, TLR2, |
| | TLR1, ITGAX, HCK, TLR8, SLC11A1, CD47, |
| | CD14, CLEC4E, CLEC7A, FCAR, FCN1, LILRA5, |
| | LILRB2, LYZ, NFAM1, P2RY13, S100A8, |
| | S100A9, SERPINA1, SIRPA, SIRPB2, TREM1, |
| | CLEC5A, CSF1, CYBB, FCGR1A, MARCO, |
| | NLRP3, FPR1, FPR3, CCL3, DAB2, OLR1, |
| | C5AR1, TREM2, MRC1, CEBPB |
| Endothelial Cell | BCL6B, CDH5, CLEC14A, CXorf36, EMCN, |
| | FAM124B, KDR, MMRN2, MYCT1, PALMD, |
| | ROBO4, SHE, TEK, TIE1 |
| Antigen Presenting Machinery (APM) | B2M, TAP1, TAP2, TAPBP, HLA-A, HLA-B, |
| | HLA-C |
| MHC2 | HLA-DRB5, HLA-DPA1, HLA-DPB1, HLA- |
| | DQB1, HLA-DRA, HLA-DRB1, HLA-DMA, HLA- |
| | DOA |
| Interferon-gamma | STAT1, CXCL9, CXCL10, CXCL11 |
| Cytotoxicity | GZMA, GZMB, GZMH, PRF1, GNLY |
| Immunoproteosome | PSMB8, PSMB9, PSMB10 |
| Apoptosis | AXIN1, BAD, BAX, BBC3, BCL2L1 |
| Inflammatory Chemokines | CCL2, CCL3, CCL4, CCL7, CCL8 |
| Hypoxia | BNIP3, SLC2A1, PGK1, BNIP3L, P4HA1, ADM, |
| | PDK1, ALDOC, PLOD2, P4HA2, MXI1 |
| MAGEs | MAGEA3, MAGEA6, MAGEA1, MAGEA12, |
| | MAGEA4, MAGEB2, MAGEC2, MAGEC1 |
| Glycolytic Activity | AKT1, HIF1A, SLC2A1, HK2, TPI1, ENO1, |
| | LDHA, PFKFB3, PFKM, GOT1, GOT2, GLUD1, |
| | HK1 |
| Interferon-downstream | IFI16, IFI27, IFI35, IFIH1, IFIT1, IFIT2, IFITM1, |
| | IFITM2, IRF1, APOL6, TMEM140, PARP9, |
| | TRIM21, GBP1, DTX3L, PSMB9, OAS1, OAS2, |
| | ISG15, MX1, IFI6, IFIT3, IRF9, STAT2 |
| Myeloid Inflammation | CXCL1, CXCL3, CXCL2, CCL20, AREG, FOSL1, |
| | CSF3, PTGS2, IER3, IL6 |

The invention provides methods for selecting patients with for treatment with immunotherapy by determining the expression level of one or more cell gene signatures (e.g., one or more of the genes listed in Table 1 or combinations thereof, e.g., as listed in Tables 2-17), and comparing the expression level of the cell gene signature to a median level for expression of the cell gene signature (e.g., the median level for expression of the cell gene signature in the cancer type), where a change in the level of expression of the cell gene signature identifies patients for treatment with therapeutic. In some embodiments, the cell gene signature is an immune cell gene signature and in another embodiment, the therapeutic is an immunotherapy. Optionally, the methods include the step of informing the patient that they have an increased likelihood of being responsive to an therapeutic and/or proving a recommendation to the patient for a particular therapeutic based on the expression level of one or more cell gene signatures (e.g., one or more of the genes listed in Table 1 or combinations thereof, e.g., as listed in Tables 2-17).

In one particular embodiment of the invention, provided is a method of selecting a treatment for a cancer patient in need thereof comprising determining the expression level of one or more genes in at least one of the signatures (a)-(q) in a biological sample obtained from the patient:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
   wherein a change in the level of expression of one or more of the genes in the at least one gene signature identifies a patient for treatment.

In another particular embodiment of the invention, provided is a method of selecting a subject having cancer for treatment with a therapeutic comprising determining the expression level of one or more genes in at least one of the signatures (a)-(q) in a biological sample obtained from the subject:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, 1L1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
   wherein a change in the level of expression of one or more of the genes in the at least one of the gene signatures (a)-(q) identifies a subject for treatment with a therapeutic.

In another particular embodiment of the invention, provided is a method of identifying a subject having cancer as likely to respond to treatment with a therapeutic comprising determining the expression level of one or more genes in at least one of the signatures (a)-(q) in a biological sample obtained from the subject:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
   wherein a change in the level of expression of one or more of the genes in the at least one of the gene signatures (a)-(q) identifies a patient likely to respond to treatment with a therapeutic.

In some embodiments, the patient is identified for treatment with a therapeutic, such as an activating immunotherapy or selected as having the likelihood of benefiting from an activating immunotherapy regimen if there is an increase in expression level of one or more cell gene signatures in the proliferation gene signature set (i.e., one or more of MKI67, CEP55, KIF2C, MELK, CENPF, EXO1, ANLN, RRM2, UBE2C, CCNB1 or CDC20). In other embodiments, the patient is identified for treatment with a suppressing immunotherapy or selected as having the likelihood of benefiting from a suppressing immunotherapy if there is a decrease in expression level of one or more cell gene signatures in the cytotoxic activity gene signature set (i.e., one or more of GZMA, GZMB, GZMH, PRF1 or GNLY). In other embodiments, in addition to determining the expression levels of one or more cell gene signatures in the proliferation and cytotoxic activity gene sets, expression levels of one or more cell gene signatures in combinations of any one of the gene sets as set forth in Tables 2-17 can be determined in order to identify a patient for a particular immunotherapy regimen (e.g., an activating immunotherapy regimen or a suppressing immunotherapy regimen). Optionally, these methods are carried out prior to administering an immunotherapy regimen in order to provide the patient with a pre-administration prognosis for response to immunotherapy.

In another embodiment of the invention, provided is a method for monitoring pharmacodynamic activity of a cancer treatment in a subject, comprising:

(i) measuring the expression level of one or more of the genes in at least one of the signatures (a)-(q) in a biological sample obtained from the subject, wherein the subject has been treated with a therapeutic,
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6; and
   (ii) determining the treatment as demonstrating pharmacodynamic activity based on the expression level of the one or more genes in the sample obtained from the subject, wherein an increased or decreased expression level of the one or more genes in the sample obtained from the subject indicates pharmacodynamic activity of the therapeutic.

In some embodiment, the patient is monitored for a pre-determined period as established by a clinician or technician performing the monitoring. In other embodiments, the patient is monitored for a pre-determined period according to standard of care.

In certain embodiments, the expression level of one or more of the genes in a cell gene signature in any one particular gene signature set from Table 1 is determined. In another embodiment, the expression levels of one or more genes in a cell gene signature in two particular gene signature sets from table 1 are determined. In some embodiments, a combination of two particular gene signature sets includes, or consists of, a combination including one or more genes of any two gene signature sets listed in Table 1. In some embodiments, a combination of two particular gene signature sets includes, or consists of, a combination including all of the genes of any two gene signature sets listed in Table 1.

In another embodiment, the expression levels of one or more of the genes in a cell gene signature in three particular gene signature sets are determined. In some embodiments, a combination of three particular gene signature sets includes, or consists of, a combination including one or more genes of any three gene signature sets listed in Table 1. In some embodiments, a combination of three particular gene signature sets includes, or consists of, a combination including all of the genes of any three gene signature sets listed in Table 1.

In another embodiment, the expression levels of one or more of the genes in a cell gene signature in four particular gene signature sets are determined. In some embodiments, a combination of four particular gene signature sets includes, or consists of, a combination including one or more genes of any four gene signature sets listed in Table 1. In some embodiments, a combination of four particular gene signature sets includes, or consists of, a combination including all of the genes of any four gene signature sets listed in Table 1.

In another embodiment, the expression levels of one or more of the genes in a cell gene signature in five particular gene signature sets are determined. In some embodiments, a combination of five particular gene signature sets includes, or consists of, a combination including one or more genes of five gene signature sets listed in Table 1. In some embodiments, a combination of five particular gene signature sets includes, or consists of, a combination including all of the genes of any five gene signature sets listed in Table 1.

In another embodiment, the expression levels of one or more of the genes in a cell gene signature in six particular gene signature sets are determined. In some embodiments, a combination of six particular gene signature sets includes, or consists of, a combination including one or more genes of any six gene signature sets listed in Table 1. In some embodiments, a combination of six particular gene signature sets includes, or consists of, a combination including all of the genes of any six gene signature sets listed in Table 1.

In another embodiment, the expression levels of one or more of the genes in a cell gene signature in seven particular gene signature sets are determined. In some embodiments, a combination of seven particular gene signature sets includes, or consists of, a combination including one or more genes of any seven gene signature sets listed in Table 1. In some embodiments, a combination of seven particular gene signature sets includes, or consists of, a combination including all of the genes of any seven gene signature sets listed in Table 1.

In another embodiment, the expression levels of one or more of the genes in a cell gene signature in eight particular gene signature sets are determined. In some embodiments, a combination of eight particular gene signature sets includes, or consists of, a combination including one or more genes of any eight gene signature sets listed in Table 1. In some embodiments, a combination of eight particular gene signature sets includes, or consists of, a combination including all of the genes of any eight gene signature sets listed in Table 1.

In another embodiment, the expression levels of one or more of the genes in a cell gene signature in nine particular gene signature sets are determined. In some embodiments, a combination of nine particular gene signature sets includes, or consists of, a combination including one or more genes of any nine gene signature sets listed in Table 1. In some embodiments, a combination of nine particular gene signature sets includes, or consists of, a combination including all of the genes of any nine gene signature sets listed in Table 1.

In another embodiment, the expression levels of one or more of the genes in a cell gene signature in ten particular gene signature sets are determined. In some embodiments, a combination of ten particular gene signature sets includes, or consists of, a combination including one or more genes of any ten gene signature sets listed in Table 1. In some embodiments, a combination of ten particular gene signature sets includes, or consists of, a combination including all of the genes of any ten gene signature sets listed in Table 1.

In another embodiment, the expression levels of one or more of the genes in a cell gene signature in eleven particular gene signature sets are determined. In some embodiments, a combination of eleven particular gene signature sets includes, or consists of, a combination including one or more genes of any eleven gene signature sets listed in Table 1. In some embodiments, a combination of eleven particular gene signature sets includes, or consists of, a combination including all of the genes of any eleven gene signature sets listed in Table 1.

In another embodiment, the expression levels of one or more of the genes in a cell gene signature in twelve particular gene signature sets are determined. In some embodiments, a combination of twelve particular gene signature sets includes, or consists of, a combination including one or more genes of any twelve gene signature sets listed in Table 1. In some embodiments, a combination of twelve particular gene signature sets includes, or consists of, a combination including all of the genes of any twelve gene signature sets listed in Table 1.

In another embodiment, the expression levels of one or more of the genes in a cell gene signature in thirteen particular gene signature sets are determined. In some embodiments, a combination of thirteen particular gene signature sets includes, or consists of, a combination including one or more genes of any thirteen gene signature sets listed in Table 1. In some embodiments, a combination of thirteen particular gene signature sets includes, or consists of, a combination including all of the genes of any thirteen gene signature sets listed in Table 1.

In another embodiment, the expression levels of one or more of the genes in a cell gene signature in fourteen particular gene signature sets are determined. In some embodiments, a combination of fourteen particular gene signature sets includes, or consists of, a combination including one or more genes of any fourteen gene signature sets listed in Table 1. In some embodiments, a combination of fourteen particular gene signature sets includes, or consists of, a combination including all of the genes of any fourteen gene signature sets listed in Table 1.

In another embodiment, the expression levels of one or more of the genes in a cell gene signature in fifteen particular gene signature sets are determined. In some embodiments, a combination of fifteen particular gene signature sets includes, or consists of, a combination including one or more genes of any fifteen gene signature sets listed in Table 1. In some embodiments, a combination of fifteen particular gene signature sets includes, or consists of, a combination including all of the genes of any fifteen gene signature sets listed in Table 1.

In another embodiment, the expression levels of one or more of the genes in a cell gene signature in sixteen particular gene signature sets are determined. In some embodiments, a combination of sixteen particular gene signature sets includes, or consists of, a combination including one or more genes of any sixteen gene signature sets listed in Table 1. In some embodiments, a combination of sixteen particular gene signature sets includes, or consists of, a combination including all of the genes of any sixteen gene signature sets listed in Table 1.

In another embodiment, the expression levels of one or more of the genes in a cell gene signature in seventeen particular gene signature sets are determined. In some embodiments, a combination of seventeen particular gene signature sets includes, or consists of, a combination including one or more genes of any seventeen gene signature sets listed in Table 1. In some embodiments, a combination of seventeen particular gene signature sets includes, or consists of, a combination including all of the genes of any seventeen gene signature sets listed in Table 1.

In one embodiment, a method provided herein is carried out using any combination of genes or any combination of gene signatures set forth in Table 1. In another embodiment, a method provided herein is carried out using any combination or permutation (in any order) of any one or more of the seventeen gene signatures set forth in Table 1. In another embodiment, a method provided herein is carried out using any combination or permutation (in any order) of the seventeen gene signatures set forth in Table 1. In another embodiment, a method provided herein is carried out using any combination or permutation (in any order) of any one or more genes of the seventeen gene signatures set forth in Table 1. In another embodiment, a method provided herein is carried out using any combination or permutation (in any order) of any one or more genes of any one or more of the seventeen gene signatures set forth in Table 1. In another embodiment, a method provided herein is carried out using any combination or permutation (in any order) of all of the genes in any one or more of the seventeen gene signatures set forth in Table 1. In another embodiment, a method provided herein is carried out using any combination or permutation (in any order) of all of the genes in all of the seventeen gene signatures set forth in Table 1.

In one particular embodiment, the expression levels of at least one gene in at least two, at least three, at least four, at least five, at least six, at least 7, at least 8 at least 9 at least 10, at least 11, at least 12, at least 13, at least 14, at least 15, at least 16 or at least 17 of the signatures (a)-(q) disclosed herein are determined in a biological sample obtained from the patient. In typical embodiments, the expression levels of at least two genes in at least one of the signatures (a)-(q) disclosed herein are determined in a biological sample obtained from the patient. In another embodiment, the expression levels of at least three genes in at least one of the signatures (a)-(q) disclosed herein are determined in a biological sample obtained from the patient. In another embodiment, the expression levels of each gene in at least one of the signatures (a)-(q) disclosed herein is determined in a biological sample obtained from the patient. In another embodiment, the expression levels of at least one gene in at least 2, at least 3, at least 4, at least 5, at least 6, at least 7, at least 8 at least 9 at least 10, at least 11, at least 12, at least 13, at least 14, at least 15, at least 16 or at least 17 of the signatures (a)-(q) disclosed herein are determined in a biological sample obtained from the patient. In another embodiment, the expression levels of at least one gene in each of the signatures (a)-(q) disclosed herein are determined in a biological sample obtained from the patient.

In one embodiment, the expression levels of each gene in each of the signatures (a)-(q) disclosed herein is determined in a biological sample obtained from the patient. In one embodiment, the expression levels of at least one gene in each of the signatures (a)-(q) disclosed herein are determined in a biological sample obtained from the patient. In other embodiments, the expression level of one or more of MKI67, CEP55, KIF2C, MELK, CENPF, EXO1, ANLN, RRM2, UBE2C, CCNB1 or CDC20 is determined in a biological sample obtained from the patient. In some embodiments, the expression level of one or more of FAP, COL6A3, ADAM12, OLFML2B, PDGFRB or LRRC32 is determined in a biological sample obtained from the patient. In some embodiments, the expression level of one or more of CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 or ICOS is determined in a biological sample obtained from the patient. In some embodiments, the expression level of one or more of ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 or CEBPB is determined in a biological sample obtained from the patient. In some embodiments, the expression level of one or more of BCL6B, CDH5, CLEC14A, CXorf36, EMCN, FAM124B, KDR, MMRN2, MYCT1, PALMD, ROBO4, SHE, TEK or TIE1 is determined in a biological sample obtained from the patient. In some embodiments, the expression level of one or more of B2M, TAP1, TAP2, TAPBP, HLA-A, HLA-B or HLA-C is determined in a biological sample obtained from the patient. In some embodiments, the expression level of one or more of HLA-DRB5, HLA-DPA1, HLA-DPB1, HLA-DQB1, HLA-DRA, HLA-DRB1, HLA-DMA or HLA-DOA is determined in a biological sample obtained from the patient. In some embodiments, the expression level of one or more of STAT1, CXCL9, CXCL10 or CXCL11 is determined in a biological sample obtained from the patient. In some embodiments, the expression level of one or more of GZMA, GZMB, GZMH, PRF1 or GNLY is determined in a biological sample obtained from the patient. In some embodiments, the expression level of one or more of PSMB8, PSMB9 or PSMB10 is determined in a biological sample obtained from the patient. In some embodiments, the expression level of one or more of AXIN1, BAD, BAX, BBC3 of BCL2L1 is determined in a biological sample obtained from the patient. In some embodiments, the expression level of one or more of CCL2, CCL3, CCL4, CCL7 or CCL8 is determined in a biological sample obtained from the patient. In some embodiments, the expression level of one or more of BNIP3, SLC2A1, PGK1, BNIP3L, P4HA1, ADM, PDK1, ALDOC, PLOD2, P4HA2 or MXI1 is determined in a biological sample obtained from the patient. In some embodiments, the expression level of one or more of MAGEA3, MAGEA6, MAGEA1, MAGEA12, MAGEA4, MAGEB2, MAGEC2 or MAGEC1 is determined in a biological sample obtained from the patient. In some embodiments, the expression level of one or more of AKT1, HIF1A, SLC2A1, HK2, TPI1, ENO1, LDHA, PFKFB3, PFKM, GOT1, GOT2, GLUD1 or HK1 is determined in a biological sample obtained from the patient. In some embodiments, the expression level of one or more of IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 or STAT2 is determined in a biological sample obtained from the patient. In some embodiments, the expression level of one or more of CXCL1, CXCL3, CXCL2, CCL20, AREG, FOSL1, CSF3, PTGS2, IER3 or IL6 is determined in a biological sample obtained from the patient.

In one embodiment, the expression level of one or more genes recited in Table 1 is linked to a biological process described herein, such as a cancer, or a condition or disease. In another embodiment, the expression level of one or more genes in at least one of the cell gene signatures recited in Table 1 is correlated to a biological process in a patient from which a biological sample has been obtained. In some embodiments, the expression level of one or more genes listed in at least the lymphoid cell gene signature recited in Table 1 is correlated with the presence or abundance of lymphoid cells in the biological sample. In some embodiments, the expression level of one or more genes listed in at least the myeloid cell gene signature recited in Table 1 is correlated with the presence or abundance of myeloid cells in the biological sample. In some embodiments, the expression level of one or more genes listed in at least the cell proliferation gene signature recited in Table 1 is correlated with cellular proliferation. In some embodiments, the expression level of one or more genes listed in at least the lymphoid cell gene signature recited in Table 1 is correlated with the presence or abundance of B cells in the biological sample. In some embodiments, the expression level of one or more genes listed in at least the lymphoid cell gene signature recited in Table 1 is correlated with the presence or abundance of Natural Killer cells in the biological sample. In some embodiments, the expression level of one or more of genes listed in at least the lymphoid cell gene signature recited in Table 1 is correlated with the presence or abundance of costimulatory ligands in the biological sample. In some embodiments, the expression level of one or more of genes listed in at least the lymphoid cell gene signature recited in Table 1 is correlated with the presence or abundance of costimulatory receptors in the biological sample. In some embodiments, the expression level of one or more of genes listed in at least the lymphoid cell gene signature recited in Table 1 is correlated with the presence or abundance of T cells in the biological sample. In some embodiments, the expression level of one or more genes listed in at least the myeloid cell gene signature listed in Table 1 is correlated with the presence or abundance of macrophage cells in the biological sample.

In some embodiments, the expression level of one or more genes listed in at least the myeloid cell gene signature recited in Table 1 is correlated with the presence or abundance of M2 macrophage cells in the biological sample. In some embodiments, the expression level of one or more of genes listed in at least the myeloid cell gene signature, the myeloid inflammation gene signature or the inflammatory chemokines gene signature recited in Table 1 is correlated with the presence or abundance of inflammatory cells in the biological sample. In some embodiments, the expression level of one or more of genes listed in at least the myeloid cell gene signature or the lymphoid cell gene signature recited in Table 1 is correlated with the presence of T cell immune blockers in the biological sample. In some embodiments, the expression level of one or more of genes listed in at least the myeloid cell gene signature or the lymphoid cell gene signature recited in Table 1 is correlated with the presence of antigen presenting cell (APC) immune blockers in the biological sample. In some embodiments, the expression level of one or more of genes listed in at least the interferon gamma gene signature or the lymphoid cell gene signature recited in Table 1 is correlated with T cell chemotaxis. In some embodiments, the expression level of one or more of genes listed in at least the antigen processing machinery (APM) cell or the immunoproteosome gene signature recited in Table 1 is correlated with the presence of antigen processing in the biological sample. In some embodiments, the expression level of one or more of genes listed in at least the cytotoxicity cell gene signature recited in Table 1 is correlated with cytolytic activity and/or the presence or abundance of cytolytic cells in the biological sample. In some embodiments, the expression level of one or more of genes listed in at least the stroma cell gene signature recited in Table 1 is correlated with the presence or abundance of active fibroblasts in the biological sample. In some embodiments, the expression level of one or more of genes listed in at least the MAGE gene signature recited in Table 1 is correlated with the presence or abundance of tumor progression in the biological sample. In some embodiments, the expression level of one or more of genes listed in at least the interferon gamma gene signature is correlated with T cell chemotaxis. In some embodiments, the expression level of one or more of genes listed in at least the apoptosis gene signature recited in Table 1 is correlated with the presence or abundance of cells undergoing apoptosis in a biological sample. In some embodiments, the expression level of one or more of genes listed in at least the hypoxia or glycolytic activity gene signature recited in Table 1 is correlated with the presence or abundance of cells initiating angiogenesis and regulating cellular metabolism to overcome hypoxia in the biological sample. In some embodiments, the expression level of one or more of genes listed in at least the interferon-downstream gene signature recited in Table 1 is correlated with the presence or abundance of cells that secrete interferon in the biological sample.

It is to be understood that a measured correlation in a biological sample to a cancer, condition or disease, according to the methods disclosed herein, is directly applicable the source from which the biological sample was obtained in the patient. For example, if the expression of one or more of the genes or biomarkers from the at least one or more gene signatures (from Table 1) are positively identified in a biological sample obtained from a tumor or tumor microenvironment, the same correlation can be made with respect to the expression of the one or more genes or biomarkers from the at least one or more gene signatures in the tumor or tumor microenvironment from which the biological sample was obtained.

In one embodiment, expression level of one or more of MKI67, CEP55, KIF2C, MELK, CENPF, EXO1, ANLN, RRM2, UBE2C, CCNB1 or CDC20 is correlated with tumor proliferation. In another embodiment, the expression level of one or more of FAP, COL6A3, ADAM12, OLFML2B, PDGFRB or LRRC32 is correlated with stromal components in a biological sample. In another embodiment, the expression level of one or more of CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 or ICOS is correlated with the lymphoid abundance and activity within a biological sample. In another embodiment, the expression level of one or more of ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 or CEBPB is correlated with the myeloid abundance and activity in a biological sample. In another embodiment, the expression level of one or more of BCL6B, CDH5, CLEC14A, CXorf36, EMCN, FAM124B, KDR, MMRN2, MYCT1, PALMD, ROBO4, SHE, TEK or TIE1 is correlated with the abundance of endothelial cells in a biological sample. In another embodiment, the expression level of one or more of B2M, TAP1, TAP2, TAPBP, HLA-A, HLA-B or HLA-C is correlated with antigen presentation and/or processing in a tumor. In another embodiment, the expression level of one or more of HLA-DRB5, HLA-DPA1, HLA-DPB1, HLA-DQB1, HLA-DRA, HLA-DRB1, HLA-DMA or HLA-DOA is correlated with the amount of class II antigen presentation in a biological sample. In another embodiment, the expression level of one or more of STAT1, CXCL9, CXCL10 or CXCL11 is correlated with interferon-gamma signaling in a biological sample. In another embodiment, the expression level of one or more of GZMA, GZMB, GZMH, PRF1 or GNLY is correlated with the amount of cytotoxic activity in a biological sample. In another embodiment, the expression level of one or more of PSMB8, PSMB9 or PSMB10 is correlated with proteasome activity in a biological sample. In another embodiment, the expression level of one or more of AXIN1, BAD, BAX, BBC3 of BCL2L1 is correlated with apoptosis in a biological sample. In another embodiment, the expression level of one or more of CCL2, CCL3, CCL4, CCL7 or CCL8 is correlated with signaling that recruits myeloid and lymphoid cells to a biological sample. In another embodiment, the expression level of one or more of BNIP3, SLC2A1, PGK1, BNIP3L, P4HA1, ADM, PDK1, ALDOC, PLOD2, P4HA2 or MXI1 is correlated with hypoxia in a biological sample. In another embodiment, the expression level of one or more of MAGEA3, MAGEA6, MAGEA1, MAGEA12, MAGEA4, MAGEB2, MAGEC2 or MAGEC1 is correlated with the presence of melanoma-associated antigens in a biological sample. In another embodiment, the expression level of one or more of AKT1, HIF1A, SLC2A1, HK2, TPI1, ENO1, LDHA, PFKFB3, PFKM, GOT1, GOT2, GLUD1 or HK1 is correlated with glycolysis in a biological sample. In another embodiment, the expression level of one or more of IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 or STAT2 is correlated with response to interferons in a biological sample. In another embodiment, the expression level of one or more of CXCL1, CXCL3, CXCL2, CCL20, AREG, FOSL1, CSF3, PTGS2, IER3 or IL6 is correlated with the presence of myeloid derived cytokines and chemokines in a biological sample.

Optionally, the methods include determining the ratio of expression levels of one or more cell gene signatures between gene sets to further identify a cancer patient for treatment with an immunotherapy or who may have the likelihood of benefiting from a particular immunotherapy. For example, the ratio of expression levels of one or more cell gene signatures in the cytotoxic activity gene set (e.g., one or more of GZMA, GZMB, GZMH, PRF1 or GNLY) may be compared to the expression levels of one or more cell gene signatures in any of the tumor proliferation set (e.g., one or more of MKI67, CEP55, KIF2C, MELK, CENPF, EXO1, ANLN, RRM2, UBE2C, CCNB1 or CDC20), to determine whether the patient should be treated with an immunotherapy or would have a likelihood of benefitting from particular immunotherapy. In other embodiments, the methods include determining the ratio of the presence of the immune cell subtype (e.g., T_{eff} to T_{reg}, T_{eff} to B cells, T_{eff} to NK cells, T_{eff} to IB T cell, T_{eff} to Immuno Blocking APC, T_{eff} to inflammatory cells) in a sample from a patient with cancer (e.g., adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma, breast cancer, lung cancer, lymphoma, melanoma, liver cancer, colorectal cancer, ovarian cancer, bladder cancer, renal cancer or gastric cancer, neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer).

The expression level of a cell gene signature may be assessed by any method known in the art suitable for determination of specific protein levels in a patient sample, including by an immunohistochemical (“IHC”) method employing antibodies specific for an immune cell gene signature (e.g. the lymphoid, cytotoxicity, MHC2, or interferon-gamma gene signatures in Table 1). Such methods are well known and routinely implemented in the art, and corresponding commercial antibodies and/or kits are readily available. In one embodiment, the expression levels of the marker/indicator proteins of the invention are assessed using the reagents and/or protocol recommendations of the antibody or kit manufacturer. The skilled person will also be aware of further means for determining the expression level of a cell gene signature disclosed herein by IHC methods.

In one embodiment, the expression level of an cell gene signature may be assessed by using nCounter® systems and methods from NanoString Technologies®, as described in US2003/0013091, US2007/0166708, US2010/0015607, US2010/0261026, US2010/0262374, US2010/01 12710, US2010/0047924, US2014/0371088, US201 1/0086774 and WO2017/015099), as a preferred means for identifying target proteins and/or target nucleic acids. nCounter® systems, and methods from NanoString Technologies® allow simultaneous multiplexed identification a plurality (800 or more) distinct target proteins and/or target nucleic acids.

Together, a comparison of the identity and abundance of the target proteins and/or target nucleic acids present in first region of interest (e.g., tissue type, a cell type (including normal and abnormal cells), and a subcellular structure within a cell) and the identity and abundance of the target proteins and/or target nucleic acids present in second region of interest or more regions of interest can be made.

The nCounter® Digital Multiplexed Immunohistochemistry (IHC) assay (see WO2017/015099) relies upon antibodies coupled to photo-cleavable oligonucleotide tags which are released from discrete regions of a tissue using focused through-objective UV (e.g., ˜365 nm) exposure. Cleaved tags are quantitated in an nCounter® assay and counts mapped back to tissue location, yielding a spatially-resolved digital profile of protein abundance. The protein-detection may be performed along with or separate from a nucleic acid-detection assay which uses nucleic acid probes comprising photo-cleavable oligonucleotide tags. Thus, this assay can provide spatially-resolved digital profile of protein abundance, spatially-resolved digital profile of protein and nucleic acid abundance, or spatially-resolved digital profile of nucleic acid abundance.

Advantages of the assay include, but are not limited to: high sensitivity (e.g., ˜1 to 4 cells), all digital counting, with large dynamic range (>10⁵), highly multiplexed (e.g., 30 targets and scalable, with no change in instrumentation, to 800 targets), simple workflow, compatibility with FFPE, no secondary antibodies (for protein detection) or amplification reagents, and potential for clinical assays.

Therefore, the expression level of one or more of the biomarkers/indicators of the invention can be routinely and reproducibly determined by a person skilled in the art without undue burden. However, to ensure accurate and reproducible results, the invention also encompasses the testing of patient samples in a specialized laboratory that can ensure the validation of testing procedures.

Furthermore, the expression level of one or more of the biomarkers/indicators of the invention can be normalized using any sensible method. For example, expression levels of the genes in any of the gene signatures in Table 1 may be normalized against housekeeping genes. Useful housekeeping genes include ABCF1, NRDE2, G6PD, OAZ1, POLR2A, SDHA, STK11IP, TBC1D10B, TBP, UBB and ZBTB34 subset combinations thereof. A useful subset of housekeeping genes which the expression levels of the genes in any of the gene signatures in Table 1 may be normalized against is ABCF1, NRDE2, G6PD, OAZ1, POLR2A, SDHA, STK11IP, TBC1D10B, TBP and UBB.

Preferably, the expression level of a cell gene signature is assessed in a biological sample that contains or is suspected to contain cancer cells. The sample may be, for example, a tissue resection, a tissue biopsy, or a metastatic lesion obtained from a patient suffering from, suspected to suffer from, or diagnosed with cancer (e.g., bladder cancer, breast cancer, colorectal cancer, gastric cancer, liver cancer, melanoma, lung cancer (e.g., non-small cell lung carcinoma), ovarian cancer, or renal cell carcinoma). Preferably, the sample is a sample of a tissue, a resection or biopsy of a tumor, a known or suspected metastatic cancer lesion or section, or a blood sample, e.g., a peripheral blood sample, known or suspected to comprise circulating cancer cells. The sample may comprise both cancer cells, i.e., tumor cells, and non-cancerous cells, and, in certain embodiments, comprises both cancerous and non-cancerous cells. In embodiments of the invention comprising the determination of gene expression in stroma components, the sample comprises both cancer/tumor cells and non-cancerous cells that are, e.g., associated with the cancer/tumor cells (e.g., tumor associated fibroblasts, endothelial cells, pericytes, the extra-cellular matrix, and/or various classes of leukocytes). In other embodiments, the skilled artisan, e.g., a pathologist, can readily discern cancer cells from non-cancerous (e.g., stromal cells, endothelial cells, etc.). Methods of obtaining biological samples including tissue resections, biopsies, and body fluids, e.g., blood samples comprising cancer/tumor cells, are well known in the art. In some embodiments, the sample obtained from the patient is collected prior to beginning any immunotherapy or other treatment regimen or therapy, e.g., chemotherapy or radiation therapy for the treatment of cancer or the management or amelioration of a symptom thereof. Therefore, in some embodiments, the sample is collected before the administration of immunotherapeutic agents or other agents, or the start of immunotherapy or other treatment regimen.

Immunohistochemical methods for assessing the expression level of one or more cell gene signatures, such as by Western blotting and ELISA-based detection may also be used in the methods of the present invention. As is understood in the art, the expression level of the biomarker/indicator proteins of the invention may also be assessed at the mRNA level by any suitable method known in the art, such as Northern blotting, real time PCR, and RT PCR. Immunohistochemical- and mRNA-based detection methods and systems are well known in the art and can be deduced from standard textbooks, such as Lottspeich (Bioanalytik, Spektrum Akademisher Verlag, 1998) or Sambrook and Russell (Molecular Cloning: A Laboratory Manual, CSH Press, Cold Spring Harbor, N.Y., U.S.A., 2001). The described methods are of particular use for determining the expression levels of a cell gene signature in a patient or group of patients relative to control levels established in a population diagnosed with advanced stages of a cancer. For use in the detection methods described herein, the skilled person has the ability to label the polypeptides or oligonucleotides encompassed by the present invention. As routinely practiced in the art, hybridization probes for use in detecting mRNA levels and/or antibodies or antibody fragments for use in IHC methods can be labeled and visualized according to standard methods known in the art. Non-limiting examples of commonly used systems include the use of radiolabels, enzyme labels, fluorescent tags, biotin-avidin complexes, chemiluminescence, and the like.

The expression level of one or more of a cell gene signature listed in Table 1 can also be determined on the protein level by taking advantage of immunoagglutination, immunoprecipitation (e.g., immunodiffusion, immunelectrophoresis, immune fixation), western blotting techniques (e.g., in situ immuno histochemistry, in situ immuno cytochemistry, affinity chromatography, enzyme immunoassays), and the like. Amounts of purified polypeptide may also be determined by physical methods, e.g., photometry. Methods of quantifying a particular polypeptide in a mixture usually rely on specific binding, e.g., of antibodies.

As mentioned above, the expression level of the biomarker/indicator proteins according to the present invention may also be reflected in increased or decreased expression of the corresponding gene(s) encoding the cell gene signature. Therefore, a quantitative assessment of the gene product prior to translation (e.g. spliced, unspliced or partially spliced mRNA) can be performed in order to evaluate the expression of the corresponding gene(s). The person skilled in the art is aware of standard methods to be used in this context or may deduce these methods from standard textbooks (e.g. Sambrook, 2001). For example, quantitative data on the respective concentration/amounts of mRNA encoding one or more of a cell gene signature as described herein can be obtained by Northern Blot, Real Time PCR, and the like.

Methods of Treatment

The invention provides methods for administering a targeted therapy to a patient having a cancer, condition or disease, where the targeted therapy may be an immunotherapy, chemotherapy, cell-based therapy (e.g. CAR-T cell), radiation, or other type of therapy or combination thereof available in the art.

The invention further provides methods for administering an activating or suppressing immunotherapy to patients with a cancer (e.g., adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma, breast cancer, lung cancer, lymphoma, melanoma, liver cancer, colorectal cancer, ovarian cancer, bladder cancer, renal cancer or gastric cancer, neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer), if the patient is determined to have a change in the level of expression of one or more cell gene signatures in any of the gene sets disclosed herein. In one embodiment, the method of the present invention comprises the step of informing the patient that they have an increased likelihood of being responsive to therapy. In another embodiment, the method of the present invention comprises the step of recommending a particular therapeutic treatment to the patient. In other embodiments, the method of the present invention further comprises the step of administering a therapy to the patient if it is determined that the patient may benefit from the therapy.

In one embodiment, the patient is administered an activating immunotherapy if there is an increase in expression level of one or more cell gene signatures in the cytotoxicity gene set (i.e., one or more of GZMA, GZMB, GZMH, PRF1, GNLY). In other embodiments, the patient is administered a suppressing immunotherapy if there is a decrease in expression level of one or more cell gene signatures in the cytotoxicity gene set (i.e., one or more of GZMA, GZMB, GZMH, PRF1, GNLY). In other embodiments, in addition to determining the expression levels of one or more cell gene signatures in the lymphoid and/or cytotoxicity gene sets, expression levels of one or more cell gene signatures in combinations of any one of the gene sets as set forth in Tables 2-17 can be determined prior to administering a particular immunotherapy regimen to the patient (e.g., an activating immunotherapy regimen or a suppressing immunotherapy regimen).

In some embodiments, the activating immunotherapy includes a GITR, OX40, TIM3, LAG3, KIR, CD28, CD137, CD27, CD40, CD70, CD276, ICOS, HVEM, NKG2D, NKG2A, MICA, 2B4 or 41BB agonist, or a combination thereof. In particular embodiments, the agonist increases, enhances, or stimulates an immune response or function in a patient having cancer. In some embodiments, the agonist modulates the expression and/or activity of a ligand (e.g., a T cell receptor ligand), and/or increases or stimulates the interaction of the ligand with its immune receptor, and/or increases or stimulates the intracellular signaling mediated by ligand binding to the immune receptor. In other embodiments, the suppressing immunotherapy includes a CTLA4, PD-1 axis, TIM3, BTLA, VISTA, LAG3, B7H4, CD96, TIGIT or a CD226 antagonist, or a combination thereof. In particular embodiments, the antagonist is an agent that inhibits and/or blocks the interaction of a ligand (e.g., a T cell receptor ligand) with its immune receptor or is an antagonist of ligand and/or receptor expression and/or activity, or is an agent that blocks the intracellular signaling mediated by a ligand (e.g., a T cell receptor ligand) with its immune receptor.

In some embodiments, the methods of the invention may further comprise administering the activating immunotherapy (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or the suppressing immunotherapy (e.g., a CTLA-4, PD-1 axis, TIM-3, BTLA, VISTA, LAG-3, B7H4, CD96, TIGIT, or CD226 antagonist or a combination thereof) with an additional therapy. The additional therapy may be radiation therapy, surgery, chemotherapy, gene therapy, DNA therapy, viral therapy, RNA therapy, bone marrow transplantation, nanotherapy, monoclonal antibody therapy, or a combination of the foregoing. The additional therapy may be in the form of an adjuvant or neoadjuvant therapy. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy may be one or more of the chemotherapeutic agents described hereinabove. For example, these methods involve the co-administration of the activating immunotherapy (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or the suppressing immunotherapy (e.g., a CTLA-4, PD-1 axis, TIM-3, BTLA, VISTA, LAG-3, B7H4, CD96, TIGIT, or CD226 antagonist or a combination thereof) with one or more additional chemotherapeutic agents (e.g., carboplatin and/or paclitaxel), as described further below. Immunotherapy optionally in combination with one or more chemotherapeutic agents (e.g., carboplatin and/or paclitaxel) preferably extends and/or improves survival, including progression free survival (PFS) and/or overall survival (OS). In one embodiment, immunotherapy extends survival at least about 20% more than survival achieved by administering an approved anti-tumor agent, or standard of care, for the cancer being treated.

In one additional embodiment, the immunotherapy comprises a checkpoint inhibitor, a chimeric antigen receptor T-cell therapy, an oncolytic vaccine, a cytokine agonist or a cytokine antagonist, or a combination thereof, or any other immunotherapy available in the art.

Oncolytic virotherapy concerns the use of lytic viruses which selectively infect and kill cancer cells. The oncolytic virus may be any oncolytic virus. Preferably it is a replication-competent virus, being replication-competent at least in the target tumor cells. In some embodiments the oncolytic virus is selected from one of an oncolytic herpes simplex virus, an oncolytic reovirus, an oncolytic vaccinia virus, an oncolytic adenovirus, an o oncolytic Newcastle Disease Virus, an oncolytic Coxsackie virus, an oncolytic measles virus. An oncolytic virus is a virus that will lyse cancer cells (oncolysis), preferably in a selective manner. Viruses that selectively replicate in dividing cells over non-dividing cells are often oncolytic. Oncolytic viruses are well known in the art and are reviewed in Molecular Therapy Vol. 18 No. 2 Feb. 2010 pg. 233-234 and are also described in WO2014/053852.

The activating immunotherapy may further comprise the use of checkpoint inhibitors. Checkpoint inhibitors are readily available in the art and include, but are not limited to, a PD-1 inhibitor, PD-L1 inhibitor, PD-L2 inhibitor, or a combination thereof.

Additionally, the immunotherapy that is provided to a patient in need thereof according to the methods of the present invention comprises providing a cytokine agonist or cytokine antagonist, that is an agonist or antagonist of interferon, IL-2, GMCSF, IL-17E, IL-6, IL-1a, IL-12, TFGB2, IL-15, IL-3, IL-13, IL-2R, IL-21, IL-4R, IL-7, M-CSF, MIF, myostatin, Il-10, Il-24, CEA, IL-11, IL-9, IL-15, IL-2Ra, TNF or a combination thereof.

For the prevention or treatment of a cancer (e.g., a cancer disclosed herein), the dose of the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist or a combination thereof) disclosed herein will depend on the type of cancer to be treated, as defined above, the severity and course of the cancer, whether the antibody is administered for preventive or therapeutic purposes, previous therapy, the patient's clinical history and response to the drug, and the discretion of the attending physician.

In one embodiment, a fixed dose of the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist or a combination thereof) is administered. The fixed dose may suitably be administered to the patient at one time or over a series of treatments. Where a fixed dose is administered, preferably it is in the range from about 20 mg to about 2000 mg. For example, the fixed dose may be approximately 420 mg, approximately 525 mg, approximately 840 mg, or approximately 1,050 mg of the agonist (e.g., a CD28, OX40, GITR, CD137, CD27, ICOS, HVEM, NKG2D, MICA, or 2B4 agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof). Where a series of doses are administered, these may, for example, be administered approximately every week, approximately every 2 weeks, approximately every 3 weeks, or approximately every 4 weeks, but preferably approximately every 3 weeks. The fixed doses may, for example, continue to be administered until disease progression, adverse event, or other time as determined by the physician. For example, from about two, three, or four, up to about 17 or more fixed doses may be administered.

In one embodiment, one or more loading dose(s) of the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist or combination thereof) or antagonist (e.g., a CTLA-4, PD-1 axis, TIM-3, BTLA, VISTA, LAG-3, B7H4, CD96, TIGIT, or CD226 antagonist, or combination thereof) are administered, followed by one or more maintenance dose(s). In another embodiment, a plurality of the same dose is administered to the patient.

While the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof) may be administered as a single anti-tumor agent, the patient is optionally treated with a combination of agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist or combination thereof) and one or more (additional) chemotherapeutic agent(s). Exemplary chemotherapeutic agents herein include: gemcitabine, carboplatin, oxaliplatin, irinotecan, fluoropyrimidine (e.g., 5-FU), paclitaxel (e.g., nab-paclitaxel), docetaxel, topotecan, capecitabine, temozolomide, interferon-alpha, and/or liposomal doxorubicin (e.g., pegylated liposomal doxorubicin). The combined administration includes co-administration or concurrent administration, using separate formulations or a single pharmaceutical formulation, and consecutive administration in either order, wherein preferably there is a time period while both (or all) active agents simultaneously exert their biological activities. Thus, the chemotherapeutic agent may be administered prior to, or following, administration of the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof). In this embodiment, the timing between at least one administration of the chemotherapeutic agent and at least one administration of the (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof) is preferably approximately 1 month or less (3 weeks, 2, weeks, 1 week, 6 days, 5, days, 4 days, 3 days, 2 days, 1 day). Alternatively, the chemotherapeutic agent and the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist or combination thereof) or antagonist (e.g., a CTLA-4, PD-1 axis, TIM-3, BTLA, VISTA, LAG-3, B7H4, CD96, TIGIT, or CD226 antagonist, or combination thereof) are administered concurrently to the patient, in a single formulation or separate formulations. Treatment with the combination of the chemotherapeutic agent (e.g., carboplatin and/or paclitaxel) and the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof) may result in a synergistic, or greater than additive, therapeutic benefit to the patient.

Particularly desired chemotherapeutic agents for combining with the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof), e.g. for therapy of ovarian cancer, include: a chemotherapeutic agent such as a platinum compound (e.g., carboplatin), a taxol such as paclitaxel or docetaxel, topotecan, or liposomal doxorubicin.

Particularly desired chemotherapeutic agents for combining with the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist or combination thereof) or antagonist (e.g., a CTLA-4, PD-1 axis, TIM-3, BTLA, VISTA, LAG-3, B7H4, CD96, TIGIT, or CD226 antagonist, or combination thereof), e.g., for therapy of breast cancer, include: chemotherapeutic agents such as capecitabine, and a taxol such as paclitaxel (e.g., nab-paclitaxel) or docetaxel.

Particularly desired chemotherapeutic agents for combining with the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof), e.g., for therapy of colorectal cancer, include: chemotherapeutic agents such as a fluoropyrimidine (e.g., 5-FU), paclitaxel, cisplatin, topotecan, irinotecan, fluoropyrimidine-oxaliplatin, fluoropyrimidine-irinotecan, FOLFOX4 (5-FU, lecovorin, oxaliplatin), and IFL (ironotecan, 5-FU, leucovorin).

Particularly desired chemotherapeutic agents for combining with the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof), e.g., for therapy of renal cell carcinoma, include: chemotherapeutic agents such as interferon-alpha2a.

A chemotherapeutic agent, if administered, is usually administered at dosages known therefore, or optionally lowered due to combined action of the drugs or negative side effects attributable to administration of the chemotherapeutic agent. Preparation and dosing schedules for such chemotherapeutic agents may be used according to manufacturers' instructions or as determined empirically by the skilled practitioner. Where the chemotherapeutic agent is paclitaxel, preferably, it is administered at a dose between about 130 mg/m² to 200 mg/m² (for example approximately 175 mg/m²), for instance, over 3 hours, once every 3 weeks. Where the chemotherapeutic agent is carboplatin, preferably it is administered by calculating the dose of carboplatin using the Calvert formula which is based on a patient's preexisting renal function or renal function and desired platelet nadir. Renal excretion is the major route of elimination for carboplatin. The use of this dosing formula, as compared to empirical dose calculation based on body surface area, allows compensation for patient variations in pretreatment renal function that might otherwise result in either underdosing (in patients with above average renal function) or overdosing (in patients with impaired renal function). The target AUC of 4-6 mg/mL/min using single agent carboplatin appears to provide the most appropriate dose range in previously treated patients. Aside from the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof) and chemotherapeutic agent, other therapeutic regimens may be combined therewith. For example, a second (third, fourth, etc.) chemotherapeutic agent(s) may be administered, wherein the second chemotherapeutic agent is an antimetabolite chemotherapeutic agent, or a chemotherapeutic agent that is not an antimetabolite. For example, the second chemotherapeutic agent may be a taxane (such as paclitaxel or docetaxel), capecitabine, or platinum-based chemotherapeutic agent (such as carboplatin, cisplatin, or oxaliplatin), anthracycline (such as doxorubicin, including, liposomal doxorubicin), topotecan, pemetrexed, vinca alkaloid (such as vinorelbine), and TLK 286.

“Cocktails” of different chemotherapeutic agents may be administered.

Other therapeutic agents that may be combined with the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist or combination thereof), and/or chemotherapeutic agent include any one or more of: a HER inhibitor, HER dimerization inhibitor (for example, a growth inhibitory HER2 antibody such as trastuzumab, or a HER2 antibody which induces apoptosis of a HER2-overexpressing cell, such as 7C2, 7F3 or humanized variants thereof); an antibody directed against a different tumor associated antigen, such as EGFR, HER3, HE R4; anti-hormonal compound, e.g., an anti-estrogen compound such as tamoxifen, or an aromatase inhibitor; a cardioprotectant (to prevent or reduce any myocardial dysfunction associated with the therapy); a cytokine; an EGFR-targeted drug (such as TARCEVA® IRESSA® or cetuximab); a tyrosine kinase inhibitor; a COX inhibitor (for instance a COX-1 or COX-2 inhibitor); non-steroidal anti-inflammatory drug, celecoxib (CELEBREX®); farnesyl transferase inhibitor (for example, Tipifarnib/ZARNESTRA® R1 15777 available from Johnson and Johnson or Lonafarnib SCH66336 available from Schering-Plough); antibody that binds oncofetal protein CA 125 such as Oregovomab (MoAb B43.13); HER2 vaccine (such as HER2AutoVac vaccine from Pharmexia, or APC8024 protein vaccine from Dendreon, or HER2 peptide vaccine from GSK/Corixa); another HER targeting therapy (e.g. trastuzumab, cetuximab, ABX-EGF, EMD7200, gefitinib, erlotinib, CP724714, CM 033, GW572016, IMC-1 1 F8, TAK165, etc); Raf and/or ras inhibitor (see, for example, WO 2003/86467); doxorubicin HCl liposome injection (DOXIL®); topoisomerase 1 inhibitor such as topotecan; taxane; HER2 and EGFR dual tyrosine kinase inhibitor such as lapatinib/GW572016; TLK286 (TELCYTA®); EMD-7200; a medicament that treats nausea such as a serotonin antagonist, steroid, or benzodiazepine; a medicament that prevents or treats skin rash or standard acne therapies, including topical or oral antibiotic; a medicament that treats or prevents diarrhea; a body temperature-reducing medicament such as acetaminophen, diphenhydramine, or meperidine; hematopoietic growth factor, etc.

Suitable dosages for any of the above-noted co-administered agents are those presently used and may be lowered due to the combined action (synergy) of the agent and the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof). In addition to the above therapeutic regimes, the patient may be subjected to surgical removal of tumors and/or cancer cells, and/or radiation therapy.

Where the agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof) is an antibody, preferably the administered antibody is a naked antibody. The agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or combination thereof) administered may be conjugated with a cytotoxic agent. Preferably, the conjugate and/or antigen to which it is bound is/are internalized by the cell, resulting in increased therapeutic efficacy of the conjugate in killing the cancer cell to which it binds. In a preferred embodiment, the cytotoxic agent targets or interferes with nucleic acid in the cancer cell. Examples of such cytotoxic agents include maytansinoids, calicheamicins, ribonucleases, and DNA endonucleases.

The agonist (e.g., a GITR, OX40, TIM3, LAG3, KIR, CD28, CD137, CD27, CD40, CD70, CD276, ICOS, HVEM, NKG2D, NKG2A, MICA, 2B4 or 41BB agonist, or combination thereof) or antagonist (e.g., a CTLA-4, PD-1 axis, TIM-3, BTLA, VISTA, LAG-3, B7H4, CD96, TIGIT, or CD226 antagonist, or a combination thereof) can be administered by gene therapy. See, for example, WO 96/07321 published Mar. 14, 1996 concerning the use of gene therapy to generate intracellular antibodies. There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus. The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Choi, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:44294432 (1 987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

A targeted therapeutic disclosed herein such as an agonist or antagonist, in which the targeted therapeutic is administered to a subject in need thereof, the targeted therapeutic includes a pharmaceutically acceptable carrier or diluent. The targeted therapeutic can be administered orally or parenterally, for example, transdermally (e.g., patch) intravenously (injection), intraperitoneally (injection), subcutaneously, and locally (injection).

Kits

This disclosure encompasses kits, which include, but are not limited to, assays, probes and directions (written instructions for their use) for determining expression levels of genes or protein levels resulting from each cell gene signature set. The components listed above can be tailored to the particular study to be undertaken. The kit can further include appropriate buffers and reagents known in the art for carrying out the necessary assays.

Any of the above aspects and embodiments can be combined with any other aspect or embodiment as disclosed here in the Summary and/or Detailed Description sections.

The following examples are presented in order to more fully illustrate the preferred embodiments of the invention. They should in no way be construed, however, as limiting the broad scope of the invention.

### EXAMPLES

### Example 1: Training if a Single Signature of Intrinsic Biology of Immune Oncology

To derive a signature measuring a given biological process, domain knowledge and literature searches is used to identify candidate genes whose expression is likely to track the process. To ensure that each signature retains strong biological plausibility, genes known to actively participate in the biological process are sought, not just genes previously reported to be correlated with it. For example, these included cytotoxicity candidate genes coding the proteins delivered by cytotoxic granules, and antigen processing candidate genes which code for the molecules used to transport antigens within the tumor and display them on the cell surface.

To screen for genes that fail to measure their intended biological process, candidate genes are tested for the co-expression patterns that would be expected from genes whose expression is linked to the biological process in question. Thus, if a collection of genes measures a process, those genes will all rise and fall as the process does and they'll be correlated. Specifically, it is required not only that candidate genes be correlated, but also that their correlation cannot be explained by another biological variable. For example, for cytotoxicity genes that are expressed in CD8 and NK cells, suggesting variable CD8 and NK cell abundance could potentially induce correlation among these genes even in the absence of any cytotoxic activity. Therefore, to believe that candidate cytotoxicity genes are measuring cytotoxicity and not merely CD8 and NK cell abundance, it is necessary for cytotoxicity signature genes to display co-expression beyond what could be explained by CD8 and NK cell abundance.

For a given set of candidate genes, the procedure for removing poorly performing genes is as follows:
1. Use biological knowledge to identify potential confounding signatures: any signatures that could plausibly explain co-expression of the candidate genes.
2. Within each of The Cancer Genome Atlas (TCGA) dataset, regress each candidate gene on the confounding signatures, and save the residuals.
3. Within each TCGA dataset, compute the correlation matrix of the signature genes' residuals, define the genes' similarity matrix as the average of these dataset-specific correlation matrices.
4. Initially define the “active” gene set as all the candidate genes in the set.
5. Over successive iterations, identify the gene with the lowest average similarity with the other genes in the active gene set, and remove it from the active gene set. Save the average similarity between the active genes at each iteration.
6. Permutation test: for 1000 random gene sets, repeat steps 2-5. Each iteration's p-value is the proportion of permutated gene sets for which the active genes at that iteration achieve a higher average similarity.
7. Choose the first iteration where the permutation p-value <0.01 and the minimum active gene's similarity with the other active genes is >0.2.

Weight Optimization

Given a set of p signature genes, the process for training optimized weights from a single dataset is as follows:

Call y_{pX1} the random vector of log₂ expression values of the p selected genes in a random patient.

Call x_{kX1} the random vector of log₂ activity levels for the process in question and the k−1 confounding processes. Let the first element of this vector represent the activity level of the process in question, and denote it x₁.

Call Σₓ the covariance of x.

Call β_{pXk} the matrix of linear associations between each process and each gene, such that β_{1,2} is the rate of increase of log₂ expression in gene 1 associated with a unit increase in the second process in x.

The signature genes' expression are modeled as follows:

Y=βx+ε,

where ε_{pX1} is the vector of errors, where var(εᵢ)=σᵢ². And write the covariance matrix of ε as Σ_{ε}=diag(σ₁², . . . , σₚ²).

Finally, call the signature weights w_{pX1}, where the signature score is calculated as w^{T}y. The w that minimizes var(w^{T}y−x₁), the variance of the difference between the signature score and the true activity level of the process in question, is what is being sought. (The mean difference is of no concern, as the unit of measurement of x₁ is undefinable.) It is further required that each element of w is positive, making each signature a simple weighted average of its expression genes. It is also required that w sums to 1, placing each signature on the log₂ scale such that a unit increase corresponds to roughly a doubling of signature gene expression.

Formally, then, following is calculated: ŵ=argmin_{w}{var(w^{T}y−x₁)} subject to w≥0 and Σᵢwᵢ=1. Now w^{T}y−x₁=w^{T}(βx+ε)−x₁=(w^{T}β+h^{T})x+w^{T}ε, where h=(1, 0, . . . , 0)^{T} such that h^{T}x=x₁. Then var(w^{T}y−x₁)=var((w^{T}β+h^{T})x+w^{T}ε)=(w^{T}β+h^{T})Σₓ(w^{T}β+h^{T})^{T}+w^{T}Σ_{ε}w=w^{T}(βΣₓβ^{T}+Σ_{ε})w+w^{T}(2βΣ_{ε}h)^{T}+h^{T}Σₓh.

As the last term is constant, ŵ is calculated as follows, ŵ=argmin_{w}{w^{T}(β Σₓβ^{T}+Σ_{ε})w+w^{T}(2βΣ_{ε}h)^{T}} subject to w≥0 and Σᵢwᵢ=1. This is a standard quadratic optimization problem, which is solved using the R library quadprog.

Before optimization, the constants in the optimization function must be estimated: Σₓ, β, and σ₁², . . . , σₚ². Estimates for all of these quantities depend on knowing the scores of the signature in question and its confounding signatures in the training dataset. As a stand-in for the unknown true level of the biological process in question, the average of the selected genes is determined, and the previously calculated scores are relied upon for the confounding signatures. Then Σₓ can be calculated as the empirical covariance matrix of these signatures scores.

Each row of β corresponds to the associations between a single gene and the biological processes under consideration. To estimate a row of β corresponding to a given gene, then, the gene's log₂ expression is regressed against signature scores for the process in question and for the confounding signatures. To avoid bias in this model, the score is re-calculated for the process in question as the average of the log₂ expression of the remaining genes, not as the average of all genes.

Finally, to obtain a gene's residual variance σⱼ², the variance of the residuals is determined from this regression model. Once these constants are defined, the quadratic optimization problem is computed and an optimal weights vector is calculated.

The above section detailed the process for estimating an optimal weights vector from a single dataset. To derive our final weights vector, the above process is applied separately to each TCGA dataset, and the average of the resulting weights vectors is determined.

Table 2 below sets forth exemplary sets of weighting coefficients generated via the process described above for use in calculating signature scores for gene signatures of the invention.

TABLE 2
Exemplary Gene Weights

| Gene Signature | Gene | Weight |
|---|---|---|
| Proliferation | MKI67 | 0.091114 |
| Proliferation | CEP55 | 0.116275 |
| Proliferation | KIF2C | 0.118987 |
| Proliferation | MELK | 0.085436 |
| Proliferation | CENPF | 0.095276 |
| Proliferation | EXO1 | 0.082624 |
| Proliferation | ANLN | 0.080802 |
| Proliferation | RRM2 | 0.081381 |
| Proliferation | UBE2C | 0.067309 |
| Proliferation | CCNB1 | 0.096929 |
| Proliferation | CDC20 | 0.083867 |
| Stroma | FAP | 0.134653 |
| Stroma | COL6A3 | 0.211119 |
| Stroma | ADAM12 | 0.112668 |
| Stroma | OLFML2B | 0.179006 |
| Stroma | PDGFRB | 0.242222 |
| Stroma | LRRC32 | 0.120331 |
| Lymphoid | CXCL10 | 0.010413 |
| Lymphoid | CXCR3 | 0.022631 |
| Lymphoid | CX3CL1 | 0.008287 |
| Lymphoid | PRF1 | 0.021885 |
| Lymphoid | GZMK | 0.015327 |
| Lymphoid | GZMB | 0.016324 |
| Lymphoid | CD27 | 0.023481 |
| Lymphoid | IL2RG | 0.023319 |
| Lymphoid | KLRK1 | 0.022768 |
| Lymphoid | CTLA4 | 0.014502 |
| Lymphoid | GZMH | 0.017586 |
| Lymphoid | CD3D | 0.028817 |
| Lymphoid | KLRB1 | 0.009325 |
| Lymphoid | KLRD1 | 0.013017 |
| Lymphoid | LCK | 0.024795 |
| Lymphoid | CD5 | 0.017805 |
| Lymphoid | IRF4 | 0.01149 |
| Lymphoid | CD8A | 0.026744 |
| Lymphoid | CD38 | 0.009396 |
| Lymphoid | EOMES | 0.012484 |
| Lymphoid | GZMM | 0.012494 |
| Lymphoid | GNLY | 0.006649 |
| Lymphoid | IFITM1 | 0.0083 |
| Lymphoid | IDO1 | 0.00774 |
| Lymphoid | MS4A1 | 0.004497 |
| Lymphoid | GZMA | 0.020973 |
| Lymphoid | CD2 | 0.041952 |
| Lymphoid | CD3E | 0.046196 |
| Lymphoid | CD3G | 0.018133 |
| Lymphoid | CD40LG | 0.010665 |
| Lymphoid | CD6 | 0.020622 |
| Lymphoid | CD7 | 0.015825 |
| Lymphoid | CD79A | 0.005826 |
| Lymphoid | CD8B | 0.011294 |
| Lymphoid | CXCL11 | 0.008773 |
| Lymphoid | CXCL13 | 0.006097 |
| Lymphoid | CXCL9 | 0.012208 |
| Lymphoid | HLA-DOB | 0.008473 |
| Lymphoid | IFNG | 0.018151 |
| Lymphoid | LAG3 | 0.014957 |
| Lymphoid | LY9 | 0.015996 |
| Lymphoid | PDCD1 | 0.018796 |
| Lymphoid | TBX21 | 0.029064 |
| Lymphoid | TIGIT | 0.030909 |
| Lymphoid | ZAP70 | 0.018452 |
| Lymphoid | SLAMF7 | 0.012334 |
| Lymphoid | CD96 | 0.030636 |
| Lymphoid | PVR | 0.024396 |
| Lymphoid | STAT1 | 0.020179 |
| Lymphoid | JAK1 | 0.025708 |
| Lymphoid | JAK2 | 0.015418 |
| Lymphoid | STAT2 | 0.031651 |
| Lymphoid | IRF9 | 0.019892 |
| Lymphoid | IGF2R | 0.015111 |
| Lymphoid | CD48 | 0.021603 |
| Lymphoid | ICOS | 0.019632 |
| Myeloid | ITGAM | 0.034733 |
| Myeloid | TLR4 | 0.018114 |
| Myeloid | IL1B | 0.013049 |
| Myeloid | CSF1R | 0.031755 |
| Myeloid | CSF3R | 0.031024 |
| Myeloid | TLR2 | 0.02849 |
| Myeloid | TLR1 | 0.014478 |
| Myeloid | ITGAX | 0.029154 |
| Myeloid | HCK | 0.048681 |
| Myeloid | TLR8 | 0.022877 |
| Myeloid | SLC11A1 | 0.032729 |
| Myeloid | CD47 | 0.029953 |
| Myeloid | CD14 | 0.038081 |
| Myeloid | CLEC4E | 0.013908 |
| Myeloid | CLEC7A | 0.032998 |
| Myeloid | FCAR | 0.024558 |
| Myeloid | FCN1 | 0.012618 |
| Myeloid | LILRA5 | 0.022702 |
| Myeloid | LILRB2 | 0.046666 |
| Myeloid | LYZ | 0.010314 |
| Myeloid | NFAM1 | 0.03044 |
| Myeloid | P2RY13 | 0.01101 |
| Myeloid | S100A8 | 0.013836 |
| Myeloid | S100A9 | 0.015231 |
| Myeloid | SERPINA1 | 0.01047 |
| Myeloid | SIRPA | 0.022067 |
| Myeloid | SIRPB2 | 0.025276 |
| Myeloid | TREM1 | 0.018972 |
| Myeloid | CLEC5A | 0.025164 |
| Myeloid | CSF1 | 0.014595 |
| Myeloid | CYBB | 0.036902 |
| Myeloid | FCGR1A | 0.021665 |
| Myeloid | MARCO | 0.009061 |
| Myeloid | NLRP3 | 0.026562 |
| Myeloid | FPR1 | 0.026696 |
| Myeloid | FPR3 | 0.025551 |
| Myeloid | CCL3 | 0.014343 |
| Myeloid | DAB2 | 0.015733 |
| Myeloid | OLR1 | 0.012732 |
| Myeloid | C5AR1 | 0.033396 |
| Myeloid | TREM2 | 0.016772 |
| Myeloid | MRC1 | 0.013418 |
| Myeloid | CEBPB | 0.023226 |
| Endothelial Cell | BCL6B | 0.04523 |
| Endothelial Cell | CDH5 | 0.123398 |
| Endothelial Cell | CLEC14A | 0.098468 |
| Endothelial Cell | CXorf36 | 0.106952 |
| Endothelial Cell | EMCN | 0.053754 |
| Endothelial Cell | FAM124B | 0.032154 |
| Endothelial Cell | KDR | 0.043769 |
| Endothelial Cell | MMRN2 | 0.102035 |
| Endothelial Cell | MYCT1 | 0.102441 |
| Endothelial Cell | PALMD | 0.031286 |
| Endothelial Cell | ROBO4 | 0.067891 |
| Endothelial Cell | SHE | 0.048303 |
| Endothelial Cell | TEK | 0.054209 |
| Endothelial Cell | TIE1 | 0.090109 |
| Antigen Presenting Machinery (APM) | B2M | 0.113864 |
| Antigen Presenting Machinery (APM) | TAP1 | 0.180766 |
| Antigen Presenting Machinery (APM) | TAP2 | 0.118815 |
| Antigen Presenting Machinery (APM) | TAPBP | 0.129885 |
| Antigen Presenting Machinery (APM) | HLA-A | 0.138324 |
| Antigen Presenting Machinery (APM) | HLA-B | 0.167481 |
| Antigen Presenting Machinery (APM) | HLA-C | 0.150865 |
| MHC2 | HLA-DRB5 | 0.071544 |
| MHC2 | HLA-DPA1 | 0.157085 |
| MHC2 | HLA-DPB1 | 0.166988 |
| MHC2 | HLA-DQB1 | 0.073489 |
| MHC2 | HLA-DRA | 0.166587 |
| MHC2 | HLA-DRB1 | 0.18042 |
| MHC2 | HLA-DMA | 0.103877 |
| MHC2 | HLA-DOA | 0.080009 |
| Interferon-gamma | STAT1 | 0.261104 |
| Interferon-gamma | CXCL9 | 0.188978 |
| Interferon-gamma | CXCL10 | 0.308838 |
| Interferon-gamma | CXCL11 | 0.24108 |
| Cytotoxicity | GZMA | 0.226344 |
| Cytotoxicity | GZMB | 0.198289 |
| Cytotoxicity | GZMH | 0.180784 |
| Cytotoxicity | PRF1 | 0.237575 |
| Cytotoxicity | GNLY | 0.157007 |
| Immunoproteosome | PSMB8 | 0.397488 |
| Immunoproteosome | PSMB9 | 0.318256 |
| Immunoproteosome | PSMB10 | 0.284256 |
| Apoptosis | AXIN1 | 0.203918 |
| Apoptosis | BAD | 0.18699 |
| Apoptosis | BAX | 0.249206 |
| Apoptosis | BBC3 | 0.192091 |
| Apoptosis | BCL2L1 | 0.167796 |
| Inflammatory Chemokines | CCL2 | 0.197584 |
| Inflammatory Chemokines | CCL3 | 0.205297 |
| Inflammatory Chemokines | CCL4 | 0.23028 |
| Inflammatory Chemokines | CCL7 | 0.155351 |
| Inflammatory Chemokines | CCL8 | 0.211488 |
| Hypoxia | BNIP3 | 0.099679 |
| Hypoxia | SLC2A1 | 0.072022 |
| Hypoxia | PGK1 | 0.130471 |
| Hypoxia | BNIP3L | 0.119342 |
| Hypoxia | P4HA1 | 0.154173 |
| Hypoxia | ADM | 0.054241 |
| Hypoxia | PDK1 | 0.109277 |
| Hypoxia | ALDOC | 0.051235 |
| Hypoxia | PLOD2 | 0.068027 |
| Hypoxia | P4HA2 | 0.07164 |
| Hypoxia | MXI1 | 0.069893 |
| MAGEs | MAGEA3 | 0.154693 |
| MAGEs | MAGEA6 | 0.15147 |
| MAGEs | MAGEA1 | 0.112482 |
| MAGEs | MAGEA12 | 0.13496 |
| MAGEs | MAGEA4 | 0.077596 |
| MAGEs | MAGEB2 | 0.118492 |
| MAGEs | MAGEC2 | 0.121232 |
| MAGEs | MAGEC1 | 0.129074 |
| Glycolytic Activity | AKT1 | 0.076033 |
| Glycolytic Activity | HIF1A | 0.071693 |
| Glycolytic Activity | SLC2A1 | 0.054196 |
| Glycolytic Activity | HK2 | 0.062052 |
| Glycolytic Activity | TPI1 | 0.100451 |
| Glycolytic Activity | ENO1 | 0.101153 |
| Glycolytic Activity | LDHA | 0.106651 |
| Glycolytic Activity | PFKFB3 | 0.066591 |
| Glycolytic Activity | PFKM | 0.057343 |
| Glycolytic Activity | GOT1 | 0.061029 |
| Glycolytic Activity | GOT2 | 0.092339 |
| Glycolytic Activity | GLUD1 | 0.058242 |
| Glycolytic Activity | HK1 | 0.092228 |
| Interferon-downstream | IFI16 | 0.025849 |
| Interferon-downstream | IFI27 | 0.026465 |
| Interferon-downstream | IFI35 | 0.052622 |
| Interferon-downstream | IFIH1 | 0.040208 |
| Interferon-downstream | IFIT1 | 0.037882 |
| Interferon-downstream | IFIT2 | 0.032315 |
| Interferon-downstream | IFITM1 | 0.033252 |
| Interferon-downstream | IFITM2 | 0.025157 |
| Interferon-downstream | IRF1 | 0.038673 |
| Interferon-downstream | APOL6 | 0.032011 |
| Interferon-downstream | TMEM140 | 0.036513 |
| Interferon-downstream | PARP9 | 0.053613 |
| Interferon-downstream | TRIM21 | 0.054735 |
| Interferon-downstream | GBP1 | 0.028901 |
| Interferon-downstream | DTX3L | 0.046913 |
| Interferon-downstream | PSMB9 | 0.038147 |
| Interferon-downstream | OAS1 | 0.044569 |
| Interferon-downstream | OAS2 | 0.055781 |
| Interferon-downstream | ISG15 | 0.03628 |
| Interferon-downstream | MX1 | 0.044668 |
| Interferon-downstream | IFI6 | 0.032674 |
| Interferon-downstream | IFIT3 | 0.064899 |
| Interferon-downstream | IRF9 | 0.067692 |
| Interferon-downstream | STAT2 | 0.050182 |
| Myeloid Inflammation | CXCL1 | 0.092222 |
| Myeloid Inflammation | CXCL3 | 0.152267 |
| Myeloid Inflammation | CXCL2 | 0.151529 |
| Myeloid Inflammation | CCL20 | 0.060025 |
| Myeloid Inflammation | AREG | 0.064212 |
| Myeloid Inflammation | FOSL1 | 0.089301 |
| Myeloid Inflammation | CSF3 | 0.090233 |
| Myeloid Inflammation | PTGS2 | 0.070274 |
| Myeloid Inflammation | IER3 | 0.132017 |
| Myeloid Inflammation | IL6 | 0.097919 |

### Training of all Signatures

The first step was to train signatures of the high-level biology likely to influence large numbers of genes but unlikely to be driven by other signatures under consideration: stroma abundance and tumor proliferation. To avoid spurious co-expression induced by batch effects or strong biological effects like subtypes, these signature genes conditional on the first three principal components of all our initial candidate genes in principal components of immune-related genes each TCGA dataset, are evaluated. The choice to perform Principal Component Analysis (PCA) on just the 1699 candidate genes and not the whole transcriptome was arbitrary but likely to be conservative, as principal components of genes relevant to immune oncology are more likely to explain variance of immune oncology gene clusters than principal components fit to more distal biology. All other signatures are trained including stroma, proliferation, and the data's first 3 principal components among their confounding variables.

The next step was to train the broadest-scope immune signatures: those of lymphoid and myeloid cell activity. This pair of signatures forms the only cycle in our hierarchy of signature dependencies: each is included as a confounding signature for the other. To reconcile these two signatures' mutual dependency, initial versions of the lymphoid and myeloid signatures are calculated as the simple mean of all their candidate genes' log₂ expression, those initial signatures are included as confounders when training the final myeloid and lymphoid signatures. All the remaining signatures include the lymphoid and myeloid signatures among their confounders. The remaining signatures have diverse additional dependencies: on signatures of immune cell type abundance and on each other. Table 3 graphs the full conditioning relationships among the signatures.

TABLE 3
Conditioning relationships among signatures.

| | Conditioned | | Conditioned | | Conditioned | | Conditioned |
|---|---|---|---|---|---|---|---|
| Signature | on | Signature | on | Signature | on | Signature | on |
| prolif | PC1 | cytotoxicity | NK cells | CD8. | CD8 T cells | BATF3.DC. | prolif |
| | | | | exhaustion | | recruitment | |
| prolif | PC2 | cytotoxicity | NK CD56dim | immunoproteasome | PC1 | BATF3.DC. | stroma |
| | | | cells | | | recruitment | |
| prolif | PC3 | cytotoxicity | prolif | immunoproteasome | PC2 | BATF3.DC. | DC |
| | | | | | | recruitment | |
| stroma | PC1 | cytotoxicity | stroma | immunoproteasome | PC3 | Inflammatory. | PC1 |
| | | | | | | chemokines | |
| stroma | PC2 | Type1.IFN | PC1 | immunoproteasome | lymphoid | Inflammatory. | PC2 |
| | | | | | | chemokines | |
| stroma | PC3 | Type1.IFN | PC2 | immunoproteasome | myeloid | Inflammatory. | PC3 |
| | | | | | | chemokines | |
| lymphoid | PC1 | Type1.IFN | PC3 | immunoproteasome | prolif | Inflammatory. | lymphoid |
| | | | | | | chemokines | |
| lymphoid | PC2 | Type1.IFN | lymphoid | immunoproteasome | stroma | Inflammatory. | myeloid |
| | | | | | | chemokines | |
| lymphoid | PC3 | Type1.IFN | myeloid | immunoproteasome | monocytic.up | Inflammatory. | prolif |
| | | | | | | chemokines | |
| lymphoid | stroma | Type1.IFN | prolif | immunoproteasome | Macrophages | Inflammatory. | stroma |
| | | | | | | chemokines | |
| lymphoid | myl.temp | Type1.IFN | stroma | immunoproteasome | Neutrophils | Hypoxia | PC1 |
| myeloid | PC1 | costim. | PC1 | immunoproteasome | DC | Hypoxia | PC2 |
| | | coinhib | | | | | |
| myeloid | PC2 | costim. | PC2 | immunoproteasome | APM | Hypoxia | PC3 |
| | | coinhib | | | | | |
| myeloid | PC3 | costim. | PC3 | immunoproteasome | MHC2 | Hypoxia | lymphoid |
| | | coinhib | | | | | |
| myeloid | stroma | costim. | lymphoid | Apoptosis | PC1 | Hypoxia | myeloid |
| | | coinhib | | | | | |
| myeloid | lym.temp | costim. | myeloid | Apoptosis | PC2 | Hypoxia | prolif |
| | | coinhib | | | | | |
| Endothelial. | PC1 | costim. | prolif | Apoptosis | PC3 | Hypoxia | stroma |
| cells | | coinhib | | | | | |
| Endothelial. | PC2 | costim. | stroma | Apoptosis | lymphoid | MAGEs | PC1 |
| cells | | coinhib | | | | | |
| Endothelial. | PC3 | costim. | T-cells | Apoptosis | myeloid | MAGEs | PC2 |
| cells | | coinhib | | | | | |
| Endothelial. | stroma | costim. | CD8 T cells | Apoptosis | prolif | MAGEs | PC3 |
| cells | | coinhib | | | | | |
| Endothelial. | lymphoid | costim | PC1 | Apoptosis | stroma | MAGEs | lymphoid |
| cells | | | | | | | |
| Endothelial. | myeloid | costim | PC2 | Tumeh. | PC1 | MAGEs | myeloid |
| cells | | | | eosinophil | | | |
| APM | PC1 | costim | PC3 | Tumeh. | PC2 | MAGEs | prolif |
| | | | | eosinophil | | | |
| APM | PC2 | costim | lymphoid | Tumeh. | PC3 | MAGEs | stroma |
| | | | | eosinophil | | | |
| APM | PC3 | costim | myeloid | Tumeh. | lymphoid | glycolytic. | PC1 |
| | | | | eosinophil | | activity | |
| APM | lymphoid | costim | prolif | Tumeh. | myeloid | glycolytic. | PC2 |
| | | | | eosinophil | | activity | |
| APM | myeloid | costim | stroma | Tumeh. | prolif | glycolytic. | PC3 |
| | | | | eosinophil | | activity | |
| APM | prolif | costim | T-cells | Tumeh. | stroma | glycolytic. | lymphoid |
| | | | | eosinophil | | activity | |
| APM | stroma | costim | CD8 T cells | gluconeogenesis | PC1 | glycolytic. | myeloid |
| | | | | | | activity | |
| MHC2 | PC1 | coinhib | PC1 | gluconeogenesis | PC2 | glycolytic. | prolif |
| | | | | | | activity | |
| MHC2 | PC2 | coinhib | PC2 | gluconeogenesis | PC3 | glycolytic. | stroma |
| | | | | | | activity | |
| MHC2 | PC3 | coinhib | PC3 | gluconeogenesis | lymphoid | IFN. | PC1 |
| | | | | | | downstream | |
| MHC2 | lymphoid | coinhib | lymphoid | gluconeogenesis | myeloid | IFN. | PC2 |
| | | | | | | downstream | |
| MHC2 | myeloid | coinhib | myeloid | gluconeogenesis | prolif | IFN. | PC3 |
| | | | | | | downstream | |
| MHC2 | DC | coinhib | prolif | gluconeogenesis | stroma | IFN. | lymphoid |
| | | | | | | downstream | |
| MHC2 | Macrophages | coinhib | stroma | Monocyte. | PC1 | IFN. | myeloid |
| | | | | MDSC. | | downstream | |
| | | | | migration.to. | | | |
| | | | | tumors | | | |
| MHC2 | B-cells | coinhib | T-cells | Monocyte. | PC2 | IFN. | prolif |
| | | | | MDSC. | | downstream | |
| | | | | migration.to. | | | |
| | | | | tumors | | | |
| MHC2 | prolif | coinhib | CD8 T cells | Monocyte. | PC3 | IFN. | stroma |
| | | | | MDSC. | | downstream | |
| | | | | migration.to. | | | |
| | | | | tumors | | | |
| MHC2 | stroma | monocytic.up | PC1 | Monocyte. | lymphoid | IFN. | IFN.gamma |
| | | | | MDSC. | | downstream | |
| | | | | migration.to. | | | |
| | | | | tumors | | | |
| IFN.gamma | PC1 | monocytic.up | PC2 | Monocyte. | myeloid | IFN. | Macrophages |
| | | | | MDSC. | | downstream | |
| | | | | migration.to. | | | |
| | | | | tumors | | | |
| IFN.gamma | PC2 | monocytic.up | PC3 | Monocyte. | prolif | IFN. | Neutrophils |
| | | | | MDSC. | | downstream | |
| | | | | migration.to. | | | |
| | | | | tumors | | | |
| IFN.gamma | PC3 | monocytic.up | lymphoid | Monocyte. | stroma | IFN. | CD8 T cells |
| | | | | MDSC. | | downstream | |
| | | | | migration.to. | | | |
| | | | | tumors | | | |
| IFN.gamma | lymphoid | monocytic.up | myeloid | Monocyte. | Monocytic.up | IFN. | Th1 cells |
| | | | | MDSC. | | downstream | |
| | | | | migration.to. | | | |
| | | | | tumors | | | |
| IFN.gamma | myeloid | monocytic.up | prolif | Monocyte. | Macrophages | Myeloid. | PC1 |
| | | | | MDSC. | | inflam | |
| | | | | migration.to. | | | |
| | | | | tumors | | | |
| IFN.gamma | NK cells | monocytic.up | stroma | Monocyte. | Neutrophils | Myeloid. | PC2 |
| | | | | MDSC. | | inflam | |
| | | | | migration.to. | | | |
| | | | | tumors | | | |
| IFN.gamma | NK CD56dim | monocytic.up | Macrophages | Monocyte. | DC | Myeloid. | PC3 |
| | cells | | | MDSC. | | inflam | |
| | | | | migration.to. | | | |
| | | | | tumors | | | |
| IFN.gamma | Th1 cells | monocytic.up | Neutrophils | Augophagy. | PC1 | Myeloid. | lymphoid |
| | | | | PTEN. | | inflam | |
| | | | | resistance | | | |
| IFN.gamma | prolif | MDSC | PC1 | Augophagy. | PC2 | Myeloid. | myeloid |
| | | | | PTEN. | | inflam | |
| | | | | resistance | | | |
| IFN.gamma | stroma | MDSC | PC2 | Augophagy. | PC3 | Myeloid. | prolif |
| | | | | PTEN. | | inflam | |
| | | | | resistance | | | |
| STAT1. | PC1 | MDSC | PC3 | Augophagy. | lymphoid | Myeloid. | stroma |
| regulated | | | | PTEN. | | inflam | |
| | | | | resistance | | | |
| STAT1. | PC2 | MDSC | lymphoid | Augophagy. | myeloid | Myeloid. | Macrophages |
| regulated | | | | PTEN. | | inflam | |
| | | | | resistance | | | |
| STAT1. | PC3 | MDSC | myeloid | Augophagy. | prolif | Myeloid. | Neutrophils |
| regulated | | | | PTEN. | | inflam | |
| | | | | resistance | | | |
| STAT1. | lymphoid | MDSC | prolif | Augophagy. | stroma | angiogenesis | PC1 |
| regulated | | | | PTEN. | | | |
| | | | | resistance | | | |
| STAT1. | myeloid | MDSC | stroma | Beta.catenin | PC1 | angiogenesis | PC2 |
| regulated | | | | | | | |
| STAT1. | NK cells | MDSC | Macrophages | Beta.catenin | PC2 | angiogenesis | PC3 |
| regulated | | | | | | | |
| STAT1. | NK CD56dim | MDSC | monocytic.up | Beta.catenin | PC3 | angiogenesis | lymphoid |
| regulated | cells | | | | | | |
| STAT1. | Th1 cells | MDSC | Neutrophils | Beta.catenin | lymphoid | angiogenesis | myeloid |
| regulated | | | | | | | |
| STAT1. | prolif | CD8.exhaustion | PC1 | Beta.catenin | myeloid | angiogenesis | prolif |
| regulated | | | | | | | |
| STAT1. | stroma | CD8.exhaustion | PC2 | Beta.catenin | prolif | angiogenesis | stroma |
| regulated | | | | | | | |
| cytotoxicity | PC1 | CD8.exhaustion | PC3 | Beta.catenin | stroma | | |
| cytotoxicity | PC2 | CD8.exhaustion | lymphoid | BATF3.DC. | PC1 | | |
| | | | | recruitment | | | |
| cytotoxicity | PC3 | CD8.exhaustion | myeloid | BATF3.DC. | PC2 | | |
| | | | | recruitment | | | |
| cytotoxicity | lymphoid | CD8.exhaustion | prolif | BATF3.DC. | PC3 | | |
| | | | | recruitment | | | |
| cytotoxicity | myeloid | CD8.exhaustion | stroma | BATF3.DC. | lymphoid | | |
| | | | | recruitment | | | |
| cytotoxicity | CD8 T cells | CD8.exhaustion | T-cells | BATF3.DC. | myeloid | | |
| | | | | recruitment | | | |

Results

Signature Training and Improved Training of Predictive Algorithms for Immunotherapy

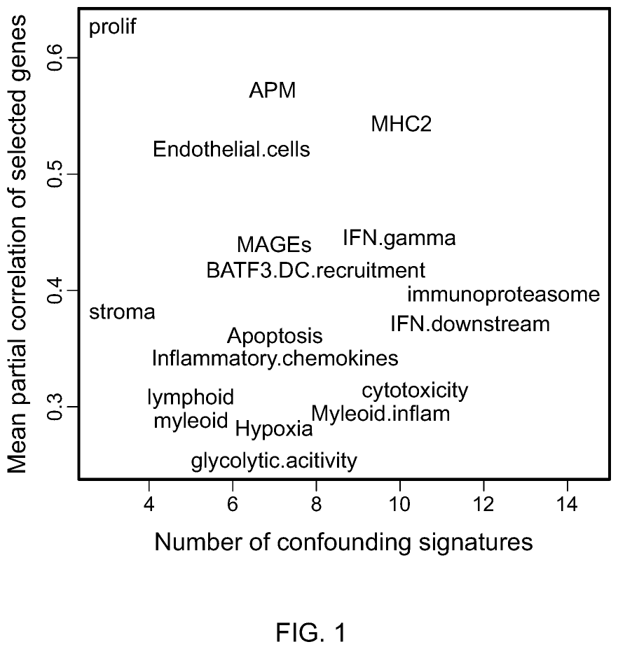

The designed method failed 12 of 31 candidate gene lists entirely; in the average passing signature, it failed 24% of the candidate genes. Table 1 displays the signatures trained and the strength of co-expression in each signature's gene set is shown in FIG. 1. Notable candidate gene lists whose co-expression was inconsistent with their measuring the target biology include CD8 exhaustion, co-stimulatory and co-inhibitory signaling, MDSC activity, and beta catenin signaling.

The small sample size typical of early phase clinical trials limits is insufficient to power a predictor training exercise using a large gene set, delaying incorporation of predictive biomarkers into trial protocols. Basing algorithm training on a small set of well-chosen signatures can improve statistical power by controlling dimensionality, focusing the training effort on the realm of biology most plausibly associated with drug response and reducing the measurement error seen in single genes.

TABLE 1
Gene Signatures

| Gene Signature | Gene Signature Gene Members |
|---|---|
| Proliferation | MKI67, CEP55, KIF2C, MELK, CENPF, EXO1, |
| | ANLN, RRM2, UBE2C, CCNB1, CDC20 |
| Stroma | FAP, COL6A3, ADAM12, OLFML2B, PDGFRB, |
| | LRRC32 |
| Lymphoid | CXCL10, CXCR3, CX3CL1, PRF1, GZMK, |
| | GZMB, CD27, IL2RG, KLRK1, CTLA4, GZMH, |
| | CD3D, KLRB1, KLRD1, LCK, CD5, IRF4, CD8A, |
| | CD38, EOMES, GZMM, GNLY, IFITM1, IDO1, |
| | MS4A1, GZMA, CD2, CD3E, CD3G, CD40LG, |
| | CD6, CD7, CD79A, CD8B, CXCL11, CXCL13, |
| | CXCL9, HLA-DOB, IFNG, LAG3, LY9, PDCD1, |
| | TBX21, TIGIT, ZAP70, SLAMF7, CD96, PVR, |
| | STAT1, JAK1, JAK2, STAT2, IRF9, IGF2R, CD48, |
| | ICOS |
| Myeloid | ITGAM, TLR4, IL1B, CSF1R, CSF3R, TLR2, |
| | TLR1, ITGAX, HCK, TLR8, SLC11A1, CD47, |
| | CD14, CLEC4E, CLEC7A, FCAR, FCN1, LILRA5, |
| | LILRB2, LYZ, NFAM1, P2RY13, S100A8, |
| | S100A9, SERPINA1, SIRPA, SIRPB2, TREM1, |
| | CLEC5A, CSF1, CYBB, FCGR1A, MARCO, |
| | NLRP3, FPR1, FPR3, CCL3, DAB2, OLR1, |
| | C5AR1, TREM2, MRC1, CEBPB |
| Endothelial Cell | BCL6B, CDH5, CLEC14A, CXorf36, EMCN, |
| | FAM124B, KDR, MMRN2, MYCT1, PALMD, |
| | ROBO4, SHE, TEK, TIE1 |
| Antigen Presenting Machinery (APM) | B2M, TAP1, TAP2, TAPBP, HLA-A, HLA-B, |
| | HLA-C |
| MHC2 | HLA-DRB5, HLA-DPA1, HLA-DPB1, HLA- |
| | DQB1, HLA-DRA, HLA-DRB1, HLA-DMA, HLA- |
| | DOA |
| Interferon-gamma | STAT1, CXCL9, CXCL10, CXCL11 |
| Cytotoxicity | GZMA, GZMB, GZMH, PRF1, GNLY |
| Immunoproteosome | PSMB8, PSMB9, PSMB10 |
| Apoptosis | AXIN1, BAD, BAX, BBC3, BCL2L1 |
| Inflammatory Chemokines | CCL2, CCL3, CCL4, CCL7, CCL8 |
| Hypoxia | BNIP3, SLC2A1, PGK1, BNIP3L, P4HA1, ADM, |
| | PDK1, ALDOC, PLOD2, P4HA2, MXI1 |
| MAGEs | MAGEA3, MAGEA6, MAGEA1, MAGEA12, |
| | MAGEA4, MAGEB2, MAGEC2, MAGEC1 |
| Glycolytic Activity | AKT1, HIF1A, SLC2A1, HK2, TPI1, ENO1, |
| | LDHA, PFKFB3, PFKM, GOT1, GOT2, GLUD1, |
| | HK1 |
| Interferon-downstream | IFI16, IFI27, IFI35, IFIH1, IFIT1, IFIT2, IFITM1, |
| | IFITM2, IRFL APOL6, TMEM140, PARP9, |
| | TRIM21, GBP1, DTX3L, PSMB9, OAS1, OAS2, |
| | ISG15, MX1, IFI6, IFIT3, IRF9, STAT2 |
| Myeloid Inflammation | CXCL1, CXCL3, CXCL2, CCL20, AREG, FOSL1, |
| | CSF3, PTGS2, IER3, IL6 |

The effectiveness of predictor training was evaluated using single genes vs. our signatures in an immunotherapy dataset with 8 responders and 34 non-responders The effectiveness of predictor training was evaluated using single genes vs. our signatures in a dataset of melanomas biopsied prior to treatment with Ipilimumab, with 8 responders and 34 non-responders. Samples were profiled using the 770-gene NanoString PanCancer Immune panel with an additional 30 genes spiked in. The data is partitioned into 1000 train-test splits, and in each training set the elastic net is used to train predictors of response from genes only, from signatures only, and from both genes and signatures. In all models, cross-validation is used to select tuning parameters. In models with both genes and signatures, cross-validation is used to select an additional tuning parameter: a constant factor between 0.1 and 1 by which the penalties applied to the signatures are reduced, thereby increasing their weight in the resulting models. Each algorithm's performance is measured with the area under the ROC curve (AUC) in its matching test set.

### Example 2: Predicting Response to an Immunotherapy Agent

Here we demonstrate the use of these signatures to predict response to an immunotherapy agent. Pratt et al (2017) collected gene expression profiles from a variety of tumors treated with anti-PD1 immunotherapy. Using the publicly available supplemental data from this paper, we calculated the immune signatures referenced in this patent filing and compared them to responder/non-responder status.

Methods

Signatures scores were calculated using the genes available in the data and the weight derivation method described in Example 1. Table 4 provides the gene list. The response between progressive disease vs. stable disease was dichotomized, partial response and complete response. A t-test was used to compare each signature's mean value in responders vs. non-responders. To evaluate whether pairs of signatures were predictive, logistic regression predicting response from pairs of signatures was carried out along with a likelihood ratio test to determine whether a model with both signatures predicted response better than the null, intercept-only mode.

TABLE 4
Gene list.

| | | | | | | | | |
|---|---|---|---|---|---|---|---|---|
| A2M | CCL3L1 | CFB | FADD | IL11 | ITGB1 | MME | S100A12 | TNFRSF14 |
| ABCB1 | CCL4 | CFD | FAS | IL11RA | ITGB2 | MNX1 | S100A7 | TNFRSF17 |
| ABL1 | CCL5 | CFI | FCER1A | IL12A | ITGB3 | MPPED1 | S100A8 | TNFRSF18 |
| ADA | CCL7 | CFP | FCER1G | IL12B | ITGB4 | MR1 | S100B | TNFRSF1A |
| ADORA2A | CCL8 | CHIT1 | FCER2 | IL12RB1 | ITK | MRC1 | SAA1 | TNFRSF1B |
| AICDA | CCND3 | CHUK | FCGR1A | IL12RB2 | JAK1 | MS4A1 | SBNO2 | TNFRSF4 |
| AIRE | CCR1 | CKLF | FCGR2A | IL13 | JAK2 | MS4A2 | SELE | TNFRSF8 |
| AKT3 | CCR2 | CLEC4A | FCGR2B | IL13RA1 | JAK3 | MSR1 | SELL | TNFRSF9 |
| ALCAM | CCR3 | CLEC4C | FCGR3A | IL13RA2 | JAM3 | MST1R | SELPLG | TNFSF10 |
| AMBP | CCR4 | CLEC5A | FEZ1 | IL15 | KIR3DL1 | MUC1 | SEMG1 | TNFSF11 |
| AMICA1 | CCR5 | CLEC6A | FLT3 | IL15RA | KIR3DL2 | MX1 | SERPINB2 | TNFSF12 |
| ANP32B | CCR6 | CLEC7A | FLT3LG | IL16 | KIR3DL3 | MYD88 | SERPING1 | TNFSF13 |
| ANXA1 | CCR7 | CLU | FN1 | IL17A | KIR_Activating_Subgroup_1 | NCAM1 | SH2B2 | TNFSF13B |
| APOE | CCR9 | CMA1 | FOS | IL17B | KIR_Activating_Subgroup_2 | NCF4 | SH2D1A | TNFSF14 |
| APP | CCRL2 | CMKLR1 | FOXJ1 | IL17F | KIR_Inhibiting_Subgroup_1 | NCR1 | SH2D1B | TNFSF15 |
| ARG1 | CD14 | COL3A1 | FOXP3 | IL17RA | KIR_Inhibiting_Subgroup_2 | NEFL | SIGIRR | TNFSF18 |
| ARG2 | CD160 | COLEC12 | FPR2 | IL17RB | KIT | NFATC1 | SIGLEC1 | TNFSF4 |
| ATF1 | CD163 | CR1 | FUT5 | IL18 | KLRB1 | NFATC2 | SLAMF1 | TNFSF8 |
| ATF2 | CD164 | CR2 | FUT7 | IL18R1 | KLRC1 | NFATC3 | SLAMF6 | TOLLIP |
| ATG10 | CD180 | CREB1 | FYN | IL18RAP | KLRC2 | NFATC4 | SLAMF7 | TP53 |
| ATG12 | CD19 | CREB5 | GAGE1 | IL19 | KLRD1 | NFKB1 | SLC11A1 | TPSAB1 |
| ATG16L1 | CD1A | CREBBP | GATA3 | IL1A | KLRF1 | NFKB2 | SMAD2 | TPTE |
| ATG5 | CD1B | CRP | GNLY | IL1B | KLRG1 | NFKBIA | SMAD3 | TRAF2 |
| ATG7 | CD1C | CSF1 | GPI | IL1R1 | KLRK1 | NLRC5 | SMPD3 | TRAF3 |
| ATM | CD1D | CSF1R | GTF3C1 | IL1R2 | LAG3 | NLRP3 | SOCS1 | TRAF6 |
| AXL | CD1E | CSF2 | GZMA | IL1RAP | LAIR2 | NOD1 | SPA17 | TREM1 |
| BAGE | CD2 | CSF2RB | GZMB | IL1RAPL2 | LAMP1 | NOD2 | SPACA3 | TREM2 |
| BATF | CD200 | CSF3 | GZMH | IL1RL1 | LAMP2 | NOS2A | SPANXB1 | TTK |
| BAX | CD207 | CSF3R | GZMK | IL1RL2 | LAMP3 | NOTCH1 | SPINK5 | TXK |
| BCL10 | CD209 | CT45A1 | GZMM | IL1RN | LBP | NRP1 | SPN | TXNIP |
| BCL2 | CD22 | CTAG1B | HAMP | IL2 | LCK | NT5E | SPO11 | TYK2 |
| BCL2L1 | CD24 | CTAGE1 | HAVCR2 | IL21 | LCN2 | NUP107 | SPP1 | UBC |
| BCL6 | CD244 | CTCFL | HCK | IL21R | LCP1 | OAS3 | SSX1 | ULBP2 |
| BID | CD247 | CTLA4 | HLA-A | IL22 | LGALS3 | OSM | SSX4 | USP9Y |
| BIRC5 | CD27 | CTSG | HLA-B | IL22RA1 | LIF | PASD1 | ST6GAL1 | VCAM1 |
| BLK | CD274 | CTSH | HLA-C | IL22RA2 | LILRA1 | PAX5 | STAT1 | VEGFA |
| BLNK | CD276 | CTSL | HLA-DMA | IL23A | LILRA4 | PBK | STAT2 | VEGFC |
| BMI1 | CD28 | CTSS | HLA-DMB | IL23R | LILRA5 | PDCD1 | STAT3 | XCL2 |
| BST1 | CD33 | CTSW | HLA-DOB | IL24 | LILRB1 | PDCD1LG2 | STAT4 | XCR1 |
| BST2 | CD34 | CX3CL1 | HLA-DPA1 | IL25 | LILRB2 | PDGFC | STAT5B | YTHDF2 |
| BTK | CD36 | CX3CR1 | HLA-DPB1 | IL26 | LILRB3 | PDGFRB | STAT6 | ZAP70 |
| BTLA | CD37 | CXCL1 | HLA-DQA1 | IL27 | LRP1 | PECAM1 | SYCP1 | ZNF205 |
| C1QA | CD38 | CXCL10 | HLA-DQB1 | IL2RA | LRRN3 | PIK3CD | SYK | ABCF1 |
| C1QB | CD3D | CXCL11 | HLA-DRA | IL2RB | LTA | PIK3CG | SYT17 | AGK |
| C1QBP | CD3E | CXCL12 | HLA-DRB3 | IL2RG | LIB | PIN1 | TAB1 | ALAS1 |
| C1R | CD3EAP | CXCL13 | HLA-DRB4 | IL3 | LTBR | PLA2G1B | TAL1 | AMMECR1L |
| C1S | CD3G | CXCL14 | HLA-E | IL32 | LTF | PLA2G6 | TANK | CC2D1B |
| C2 | CD4 | CXCL16 | HLA-G | IL34 | LTK | PLAU | TAP1 | CNOT10 |
| C3 | CD40 | CXCL2 | HMGB1 | IL3RA | LY86 | PLAUR | TAP2 | CNOT4 |
| C3AR1 | CD40LG | CXCL3 | HRAS | IL4 | LY9 | PMCH | TAPBP | COG7 |
| C4B | CD44 | CXCL5 | HSD11B1 | IL4R | LY96 | PNMA1 | TARP | DDX50 |
| C4BPA | CD46 | CXCL6 | ICAM1 | IL5 | LYN | POU2AF1 | TBK1 | DHX16 |
| C5 | CD47 | CXCL9 | ICAM2 | IL5RA | MAF | POU2F2 | TBX21 | DNAJC14 |
| C6 | CD48 | CXCR1 | ICAM3 | IL6 | MAGEA1 | PPARG | TCF7 | EDC3 |
| C7 | CD5 | CXCR2 | ICAM4 | IL6R | MAGEA12 | PPBP | TFE3 | EIF2B4 |
| C8A | CD53 | CXCR3 | ICOS | IL6ST | MAGEA3 | PRAME | TFEB | ERCC3 |
| C8B | CD55 | CXCR4 | ICOSLG | IL7 | MAGEA4 | PRF1 | TFRC | FCF1 |
| C8G | CD58 | CXCR5 | IDO1 | IL7R | MAGEB2 | PRG2 | TGFB1 | G6PD |
| C9 | CD59 | CXCR6 | IFI16 | IL8 | MAGEC1 | PRKCD | TGFB2 | GPATCH3 |
| CAMP | CD6 | CYBB | IFI27 | IL9 | MAGEC2 | PRKCE | THBD | GUSB |
| CARD11 | CD63 | CYFIP2 | IFI35 | ILF3 | MAP2K1 | PRM1 | THBS1 | HDAC3 |
| CARD9 | CD68 | CYLD | IFIH1 | INPP5D | MAP2K2 | PSEN1 | THY1 | HPRT1 |
| CASP1 | CD7 | DDX43 | IFIT1 | IRAK1 | MAP2K4 | PSEN2 | TICAM1 | MRPS5 |
| CASP10 | CD70 | DDX58 | IFIT2 | IRAK2 | MAP3K1 | PSMB10 | TICAM2 | MTMR14 |
| CASP3 | CD74 | DEFB1 | IFITM1 | IRAK4 | MAP3K5 | PSMB7 | TIGIT | NOL7 |
| CASP8 | CD79A | DMBT1 | IFITM2 | IRF1 | MAP3K7 | PSMB8 | TIRAP | NUBP1 |
| CCL1 | CD79B | DOCK9 | IFNA1 | IRF2 | MAP4K2 | PSMB9 | TLR1 | POLR2A |
| CCL11 | CD80 | DPP4 | IFNA17 | IRF3 | MAPK1 | PSMD7 | TLR10 | PPIA |
| CCL13 | CD81 | DUSP4 | IFNA2 | IRF4 | MAPK11 | PTGDR2 | TLR2 | PRPF38A |
| CCL14 | CD83 | DUSP6 | IFNA7 | IRF5 | MAPK14 | PTGS2 | TLR3 | SAP130 |
| CCL15 | CD84 | EBI3 | IFNA8 | IRF7 | MAPK3 | PTPRC | TLR4 | SDHA |
| CCL16 | CD86 | ECSIT | IFNAR1 | IRF8 | MAPK8 | PVR | TLR5 | SF3A3 |
| CCL17 | CD8A | EGR1 | IFNAR2 | IRGM | MAPKAPK2 | PYCARD | TLR6 | TBP |
| CCL18 | CD8B | EGR2 | IFNB1 | ISG15 | MARCO | RAG1 | TLR7 | TLK2 |
| CCL19 | CD9 | ELANE | IFNG | ISG20 | MASP1 | REL | TLR8 | TMUB2 |
| CCL2 | CD96 | ELK1 | IFNGR1 | ITCH | MASP2 | RELA | TLR9 | TRIM39 |
| CCL20 | CD97 | ENG | IFNL1 | ITGA1 | MAVS | RELB | TMEFF2 | TUBB |
| CCL21 | CD99 | ENTPD1 | IFNL2 | ITGA2 | MBL2 | REPS1 | TNF | USP39 |
| CCL22 | CDH1 | EOMES | IGF1R | ITGA2B | MCAM | RIPK2 | TNFAIP3 | ZC3H14 |
| CCL23 | CDH5 | EP300 | IGF2R | ITGA4 | MEF2C | ROPN1 | TNFRSF10B | ZKSCAN5 |
| CCL24 | CDK1 | EPCAM | IGLL1 | ITGA5 | MEFV | RORA | TNFRSF10C | ZNF143 |
| CCL25 | CDKN1A | ETS1 | IKBKB | ITGA6 | MERTK | RORC | TNFRSF11A | ZNF346 |
| CCL26 | CEACAM1 | EWSR1 | IKBKE | ITGAE | MFGE8 | RPS6 | TNFRSF11B | |
| CCL27 | CEACAM6 | F12 | IKBKG | ITGAL | MICA | RRAD | TNFRSF12A | |
| CCL28 | CEACAM8 | F13A1 | IL10 | ITGAM | MICB | RUNX1 | TNFRSF13B | |
| CCL3 | CEBPB | F2RL1 | IL10RA | ITGAX | MIF | RUNX3 | TNFRSF13C | |

Results

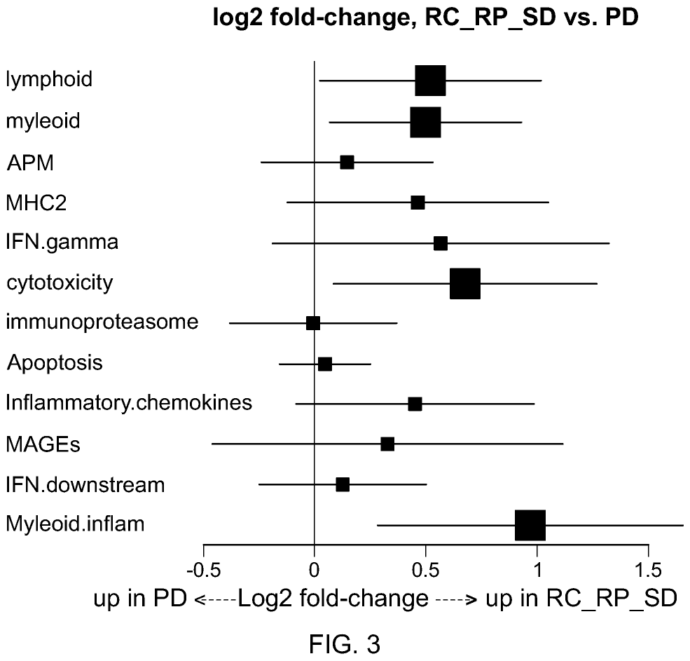

Many of the immune gene signatures are associated with response (FIG. 3), showing the ability of these signatures to predict immunotherapy response before it is clinically apparent.

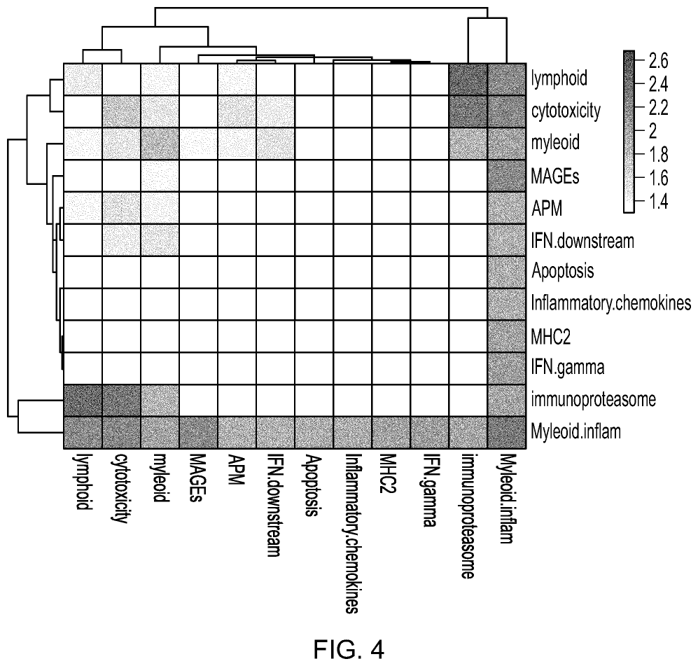

Many pairs of immune signatures were also associated with anti-PD1 response in this data (FIG. 4).

### CONCLUSIONS

The immune signatures described here can be used individually or in combination to predict immunotherapy response.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A method of selecting a treatment for a cancer patient in need thereof comprising determining the expression level of one or more genes in at least one of the signatures (a)-(q) in a biological sample obtained from the patient:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
wherein a change in the level of expression of one or more of the genes in the at least one gene signature identifies a patient for treatment.

2. The method of claim 1, wherein the expression levels of at least two genes in at least one of the signatures (a)-(q) are determined in a biological sample obtained from the patient.

3. The method of claim 1, wherein the expression levels of at least three genes in at least one of the signatures (a)-(q) are determined in a biological sample obtained from the patient.

4. The method of claim 1, wherein the expression levels of each gene in at least one of the signatures (a)-(q) is determined in a biological sample obtained from the patient.

5. The method of claim 1, wherein the expression levels of at least one gene in at least two, at least three, at least four, at least five, at least six, at least 7, at least 8 at least 9 at least 10, at least 11, at least 12, at least 13, at least 14, at least 15 or at least 16 of the signatures (a)-(q) are determined in a biological sample obtained from the patient.

6. The method of claim 1, wherein the expression levels of at least one gene in each of the signatures (a)-(q) are determined in a biological sample obtained from the patient.

7. The method of claim 1, wherein the expression levels of each gene in each of the signatures (a)-(q) are determined in a biological sample obtained from the patient.

8. The method of claim 1, wherein the expression level of one or more of MKI67, CEP55, KIF2C, MELK, CENPF, EXO1, ANLN, RRM2, UBE2C, CCNB1 or CDC20 is determined in a biological sample obtained from the patient.

9. The method of claim 1, wherein the expression level of one or more of FAP, COL6A3, ADAM12, OLFML2B, PDGFRB or LRRC32 is determined in a biological sample obtained from the patient.

10. The method of claim 1, wherein the expression level of one or more of CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 or ICOS is determined in a biological sample obtained from the patient.

11. The method of claim 1, wherein the expression level of one or more of ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 or CEBPB is determined in a biological sample obtained from the patient.

12. The method of claim 1, wherein the expression level of one or more of BCL6B, CDH5, CLEC14A, CXorf36, EMCN, FAM124B, KDR, MMRN2, MYCT1, PALMD, ROBO4, SHE, TEK or TIE1 is determined in a biological sample obtained from the patient.

13. The method of claim 1, wherein the expression level of one or more of B2M, TAP1, TAP2, TAPBP, HLA-A, HLA-B or HLA-C is determined in a biological sample obtained from the patient.

14. The method of claim 1, wherein the expression level of one or more of HLA-DRB5, HLA-DPA1, HLA-DPB1, HLA-DQB1, HLA-DRA, HLA-DRB1, HLA-DMA or HLA-DOA is determined in a biological sample obtained from the patient.

15. The method of claim 1, wherein the expression level of one or more of STAT1, CXCL9, CXCL10 or CXCL11 is determined in a biological sample obtained from the patient.

16. The method of claim 1, wherein the expression level of one or more of GZMA, GZMB, GZMH, PRF1 or GNLY is determined in a biological sample obtained from the patient.

17. The method of claim 1, wherein the expression level of one or more of PSMB8, PSMB9 or PSMB10 is determined in a biological sample obtained from the patient.

18. The method of claim 1, wherein the expression level of one or more of AXIN1, BAD, BAX, BBC3 of BCL2L1 is determined in a biological sample obtained from the patient.

19. The method of claim 1, wherein the expression level of one or more of CCL2, CCL3, CCL4, CCL7 or CCL8 is determined in a biological sample obtained from the patient.

20. The method of claim 1, wherein the expression level of one or more of BNIP3, SLC2A1, PGK1, BNIP3L, P4HA1, ADM, PDK1, ALDOC, PLOD2, P4HA2 or MXI1 is determined in a biological sample obtained from the patient.

21. The method of claim 1, wherein the expression level of one or more of MAGEA3, MAGEA6, MAGEA1, MAGEA12, MAGEA4, MAGEB2, MAGEC2 or MAGEC1 is determined in a biological sample obtained from the patient.

22. The method of claim 1, wherein the expression level of one or more of AKT1, HIF1A, SLC2A1, HK2, TPI1, ENO1, LDHA, PFKFB3, PFKM, GOT1, GOT2, GLUD1 or HK1 is determined in a biological sample obtained from the patient.

23. The method of claim 1, wherein the expression level of one or more of IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 or STAT2 is determined in a biological sample obtained from the patient.

24. The method of claim 1, wherein the expression level of one or more of CXCL1, CXCL3, CXCL2, CCL20, AREG, FOSL1, CSF3, PTGS2, IER3 or IL6 is determined in a biological sample obtained from the patient.

25. The method of claim 1, further comprising the step of informing the patient that they have an increased likelihood of being responsive to therapy.

26. The method of claim 1 or 25, further comprising the step of recommending a particular therapeutic treatment to the patient.

27. The method of claim 1, 25 or 26, further comprising the step of administering a therapy to the patient if it is determined that the patient may benefit from the therapy.

28. The method of claim 1, 25, 26 or 27, wherein the therapy is an immunotherapy.

29. The method of claim 28, wherein the immunotherapy comprises a checkpoint inhibitor, a chimeric antigen receptor T-cell therapy, an oncolytic vaccine, a cytokine agonist or a cytokine antagonist, or a combination thereof.

30. The method of claim 28, wherein the immunotherapy comprises a PD-1 inhibitor, PD-L1 inhibitor, PD-L2 inhibitor, GITR agonist, OX40 agonist, TIM3 agonist, LAG3 agonist, KIR agonist, CD28 agonist, CD137 agonist, CD27 agonist, CD40 agonist, CD70 agonist, CD276 agonist, ICOS agonist, HVEM agonist, NKG2D agonist, NKG2A agonist, MICA agonist, 2B4 agonist, 41BB agonist, CTLA4 antagonist, PD-1 axis antagonist, TIM3 antagonist, BTLA antagonist, VISTA antagonist, LAG3 antagonist, B7H4 antagonist, CD96 antagonist, TIGIT antagonist, CD226 antagonist or a combination thereof.

31. The method of claim 29, wherein the cytokine agonist or cytokine antagonist is an agonist or antagonist of interferon, IL-2, GMCSF, IL-17E, IL-6, IL-1a, IL-12, TFGB2, IL-15, IL-3, IL-13, IL-2R, IL-21, IL-4R, IL-7, M-CSF, MIF, myostatin, Il-10, Il-24, CEA, IL-11, IL-9, IL-15, IL-2Ra, TNF or a combination thereof.

32. The method of claim 1, wherein the cancer is adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma.

33. The method of claim 1, wherein the cancer is breast cancer, lung cancer, lymphoma, melanoma, liver cancer, colorectal cancer, ovarian cancer, bladder cancer, renal cancer or gastric cancer.

34. The method of claim 1, wherein the cancer is neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

35. The method of claim 1, wherein expression of the one or more genes in the biological sample form the patient is determined by measuring mRNA.

36. The method of claim 1, wherein expression of the one or more genes in the biological sample form the patient is determined by measuring mRNA in plasma.

37. The method of claim 1, wherein expression of the one or more genes in the biological sample form the patient is determined by measuring mRNA in tissue.

38. The method of claim 1, wherein expression of the one or more genes in the biological sample form the patient is determined by measuring mRNA in FFPE tissue.

39. The method of claim 1, wherein expression of the one or more genes in the biological sample form the patient is determined by measuring protein levels.

40. The method of claim 1, wherein expression of the one or more genes in the biological sample form the patient is determined by measuring protein levels in plasma.

41. The method of claim 1, wherein expression of the one or more genes in the biological sample form the patient is determined by measuring protein levels in tissue.

42. The method of claim 1, wherein expression of the one or more genes in the biological sample form the patient is determined by measuring protein levels in FFPE tissue.

43. The method of claim 1, wherein the biological sample is tumor tissue.

44. The method of claim 1, wherein the biological sample is blood.

45. The method of claim 1, wherein the expression level of one or more of MKI67, CEP55, KIF2C, MELK, CENPF, EXO1, ANLN, RRM2, UBE2C, CCNB1 or CDC20 is correlated with tumor proliferation.

46. The method of claim 1, wherein the expression level of one or more of FAP, COL6A3, ADAM12, OLFML2B, PDGFRB or LRRC32 is correlated with stromal components in a biological sample.

47. The method of claim 1, wherein the expression level of one or more of CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 or ICOS is correlated with the lymphoid abundance and activity within a biological sample.

48. The method of claim 1, wherein the expression level of one or more of ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 or CEBPB is correlated with the myeloid abundance and activity in a biological sample.

49. The method of claim 1, wherein the expression level of one or more of BCL6B, CDH5, CLEC14A, CXorf36, EMCN, FAM124B, KDR, MMRN2, MYCT1, PALMD, ROBO4, SHE, TEK or TIE1 is correlated with the abundance of endothelial cells in a biological sample.

50. The method of claim 1, wherein the expression level of one or more of B2M, TAP1, TAP2, TAPBP, HLA-A, HLA-B or HLA-C is correlated with antigen presentation and/or processing in a tumor.

51. The method of claim 1, wherein the expression level of one or more of HLA-DRB5, HLA-DPA1, HLA-DPB1, HLA-DQB1, HLA-DRA, HLA-DRB1, HLA-DMA or HLA-DOA is correlated with the amount of class II antigen presentation in a biological sample.

52. The method of claim 1, wherein the expression level of one or more of STAT1, CXCL9, CXCL10 or CXCL11 is correlated with interferon-gamma signaling in a biological sample.

53. The method of claim 1, wherein the expression level of one or more of GZMA, GZMB, GZMH, PRF1 or GNLY is correlated with the amount of cytotoxic activity in a biological sample.

54. The method of claim 1, wherein the expression level of one or more of PSMB8, PSMB9 or PSMB10 is correlated with proteasome activity in a biological sample.

55. The method of claim 1, wherein the expression level of one or more of AXIN1, BAD, BAX, BBC3 of BCL2L1 is correlated with apoptosis in a biological sample.

56. The method of claim 1, wherein the expression level of one or more of CCL2, CCL3, CCL4, CCL7 or CCL8 is correlated with signaling that recruits myeloid and lymphoid cells to a biological sample.

57. The method of claim 1, wherein the expression level of one or more of BNIP3, SLC2A1, PGK1, BNIP3L, P4HA1, ADM, PDK1, ALDOC, PLOD2, P4HA2 or MXI1 is correlated with hypoxia in a biological sample.

58. The method of claim 1, wherein the expression level of one or more of MAGEA3, MAGEA6, MAGEA1, MAGEA12, MAGEA4, MAGEB2, MAGEC2 or MAGEC1 is correlated with the presence of melanoma-associated antigens in a biological sample.

59. The method of claim 1, wherein the expression level of one or more of AKT1, HIF1A, SLC2A1, HK2, TPI1, ENO1, LDHA, PFKFB3, PFKM, GOT1, GOT2, GLUD1 or HK1 is correlated with glycolysis in a biological sample.

60. The method of claim 1, wherein the expression level of one or more of IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 or STAT2 is correlated with response to interferons in a biological sample.

61. The method of claim 1, wherein the expression level of one or more of CXCL1, CXCL3, CXCL2, CCL20, AREG, FOSL1, CSF3, PTGS2, IER3 or IL6 is correlated with the presence of myeloid derived cytokines and chemokines in a biological sample.

62. A method of selecting a subject having cancer for treatment with a therapeutic comprising determining the expression level of one or more genes in at least one of the signatures (a)-(q) in a biological sample obtained from the subject:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMA ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
wherein a change in the level of expression of one or more of the genes in the at least one of the gene signatures (a)-(q) identifies a subject for treatment with a therapeutic.

63. A method of identifying a subject having cancer as likely to respond to treatment with a therapeutic comprising determining the expression level of one or more genes in at least one of the signatures (a)-(q) in a biological sample obtained from the subject:
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6;
wherein a change in the level of expression of one or more of the genes in the at least one of the gene signatures (a)-(q) identifies a patient likely to respond to treatment with a therapeutic.

64. A method for monitoring pharmacodynamic activity of a cancer treatment in a subject, comprising:
(i) measuring the expression level of one or more of the genes in at least one of the signatures (a)-(q) in a biological sample obtained from the subject, wherein the subject has been treated with a therapeutic
(a) MKI67, CEP55, KIF2C, MELK, CENPF, EXO1, ANLN, RRM2, UBE2C, CCNB1 and CDC20;
(b) FAP, COL6A3, ADAM12, OLFML2B, PDGFRB and LRRC32;
(c) CXCL10, CXCR3, CX3CL1, PRF1, GZMK, GZMB, CD27, IL2RG, KLRK1, CTLA4, GZMH, CD3D, KLRB1, KLRD1, LCK, CD5, IRF4, CD8A, CD38, EOMES, GZMM, GNLY, IFITM1, IDO1, MS4A1, GZMA, CD2, CD3E, CD3G, CD40LG, CD6, CD7, CD79A, CD8B, CXCL11, CXCL13, CXCL9, HLA-DOB, IFNG, LAG3, LY9, PDCD1, TBX21, TIGIT, ZAP70, SLAMF7, CD96, PVR, STAT1, JAK1, JAK2, STAT2, IRF9, IGF2R, CD48 and ICOS;
(d) ITGAM, TLR4, IL1B, CSF1R, CSF3R, TLR2, TLR1, ITGAX, HCK, TLR8, SLC11A1, CD47, CD14, CLEC4E, CLEC7A, FCAR, FCN1, LILRA5, LILRB2, LYZ, NFAM1, P2RY13, S100A8, S100A9, SERPINA1, SIRPA, SIRPB2, TREM1, CLEC5A, CSF1, CYBB, FCGR1A, MARCO, NLRP3, FPR1, FPR3, CCL3, DAB2, OLR1, C5AR1, TREM2, MRC1 and CEBPB;
(e) BCL6B, CDH5, CLEC14A, CXorf36, EMCN, FAM124B, KDR, MMRN2, MYCT1, PALMD, ROBO4, SHE, TEK and TIE1;
(f) B2M, TAP1, TAP2, TAPBP, HLA-A, HLA-B and HLA-C;
(g) HLA-DRB5, HLA-DPA1, HLA-DPB1, HLA-DQB1, HLA-DRA, HLA-DRB1, HLA-DMA and HLA-DOA;
(h) STAT1, CXCL9, CXCL10 and CXCL11;
(i) GZMA, GZMB, GZMH, PRF1 and GNLY;
(j) PSMB8, PSMB9 and PSMB10;
(k) AXIN1, BAD, BAX, BBC3 and BCL2L1;
(l) CCL2, CCL3, CCL4, CCL7 and CCL8;
(m) BNIP3, SLC2A1, PGK1, BNIP3L, P4HA1, ADM, PDK1, ALDOC, PLOD2, P4HA2 and MXI1;
(n) MAGEA3, MAGEA6, MAGEA1, MAGEA12, MAGEA4, MAGEB2, MAGEC2 and MAGEC1;
(o) AKT1, HIF1A, SLC2A1, HK2, TPI1, ENO1, LDHA, PFKFB3, PFKM, GOT1, GOT2, GLUD1 and HK1;
(p) IFI16, IFI27, IFI35, IFIH1, IFIT1, IFIT2, IFITM1, IFITM2, IRF1, APOL6, TMEM140, PARP9, TRIM21, GBP1, DTX3L, PSMB9, OAS1, OAS2, ISG15, MX1, IFI6, IFIT3, IRF9 and STAT2;
(q) CXCL1, CXCL3, CXCL2, CCL20, AREG, FOSL1, CSF3, PTGS2, IER3 and IL6; and
(ii) determining the treatment as demonstrating pharmacodynamic activity based on the expression level of the one or more genes in the sample obtained from the subject, wherein an increased or decreased expression level of the one or more genes in the sample obtained from the subject indicates pharmacodynamic activity of the therapeutic.

65. The method of claim 63 or 64 wherein the biological sample is obtained from the subject before the therapeutic is administered to the subject.

66. The method of claim 63 or 64 wherein the biological sample is obtained from the subject after the therapeutic is administered to the subject.

67. The method of any of claim 1, 62, 63 or 64, further comprising administering to the subject at least one therapeutically effective amount of at least one treatment.

68. The method of claim 67, wherein the at least one treatment comprises anti-cancer therapy.

69. The method of claim 67, wherein the at least one treatment comprises immunotherapy.

70. The method of claim 69, wherein immunotherapy comprises activating immunotherapy, suppressing immunotherapy, or a combination of an activating and a suppressing immunotherapy.

71. The method of claim 69, wherein immunotherapy comprises the administration of at least one therapeutically effective amount of at least one checkpoint inhibitor, at least one therapeutically effective amount of at least one chimeric antigen receptor T-cell therapy, at least one therapeutically effective amount of at least one oncolytic vaccine, at least one therapeutically effective amount of at least one cytokine agonist, at least one therapeutically effective amount of at least one cytokine antagonist, or any combination thereof.
